(19) United States
(12) Patent Application Publication
(10) Pub. No.: US 2005/0171037 A1
(43) Pub. Date: Aug. 4, 2005

(54) Therapeutic combinations

(75) Inventors: Evan Burr Smith, San Diego, CA (US); Jonathan Quoc Tran, San Diego, CA (US)

Correspondence Address: AGOURON PHARMACEUTICALS, INC., 10777 SCIENCE CENTER DRIVE, SAN DIEGO, CA, 92121, US

(73) Assignee: Agouron Pharmaceuticals, Inc.

(21) Appl. No.: 11/046,025

(22) Filed: Jan. 28, 2005

Related U.S. Application Data
(60) Provisional application No. 60/540,746, filed on Jan. 30, 2004.
(60) Provisional application No. 60/614,997, filed on Oct. 1, 2004.

(51) Int. Cl. A61K 31/7056; A61K 31/426

(52) U.S. Cl. 514/43; 514/369

(57) ABSTRACT

The present invention relates to methods for improving the pharmacokinetics of certain compounds useful as inhibitors of the HIV protease enzyme by inhibiting the enzyme activity of cytochrome P450. The present invention also relates to compositions comprising certain compounds useful as inhibitors of the HIV protease enzyme and at least one agent that inhibits the enzyme activity of cytochrome P450.

This application claims priority from U.S. Provisional Application Nos. 60/540,746, filed Jan. 30, 2004, and 60/614,997, filed Oct. 1, 2004, both of which are hereby incorporated by reference.

### BACKGROUND

The present invention relates to methods for improving the pharmacokinetics of certain compounds useful as inhibitors of the HIV protease enzyme by inhibiting the enzyme activity of cytochrome P450. The present invention also relates to compositions comprising certain compounds useful as inhibitors of the HIV protease enzyme and at least one agent that inhibits the enzyme activity of cytochrome P450.

Acquired Immune Deficiency Syndrome (AIDS) causes a gradual breakdown of the body's immune system as well as progressive deterioration of the central and peripheral nervous systems. Since its initial recognition in the early 1980's, AIDS has spread rapidly and has now reached epidemic proportions within a relatively limited segment of the population. Intensive research has led to the discovery of the responsible agent, human T-lymphotropic retrovirus III (HTLV-III), now more commonly referred to as the human immunodeficiency virus or HIV.

HIV is a member of the class of viruses known as retroviruses. The retroviral genome is composed of RNA which is converted to DNA by reverse transcription. This retroviral DNA is then stably integrated into a host cell's chromosome and, employing the replicative processes of the host cells, produces new retroviral particles and advances the infection to other cells. HIV appears to have a particular affinity for the human T-4 lymphocyte cell which plays a vital role in the body's immune system. HIV infection of these white blood cells depletes this white cell population. Eventually, the immune system is rendered inoperative and ineffective against various opportunistic diseases such as, among others, pneumocystic carini pneumonia, Kaposi's sarcoma, and cancer of the lymph system.

Although the exact mechanism of the formation and working of the HIV virus is not understood, identification of the virus has led to some progress in controlling the disease. For example, the drug azidothymidine (AZT) has been found effective for inhibiting the reverse transcription of the retroviral genome of the HIV virus, thus giving a measure of control, though not a cure, for patients afflicted with AIDS. The search continues for drugs that can cure or at least provide an improved measure of control of the deadly HIV virus.

Retroviral replication routinely features post-translational processing of polyproteins. This processing is accomplished by virally encoded HIV protease enzyme. This yields mature polypeptides that will subsequently aid in the formation and function of infectious virus. If this molecular processing is stifled, then the normal production of HIV is terminated. Therefore, inhibitors of HIV protease may function as anti-HIV viral agents.

HIV protease is one of the translated products from the HIV structural protein pol gene. This retroviral protease specifically cleaves other structural polypeptides at discrete sites to release these newly activated structural proteins and enzymes, thereby rendering the virion replication-competent. As such, inhibition of the HIV protease by potent compounds may prevent proviral integration of infected T-lymphocytes during the early phase of the HIV-1 life cycle, as well as inhibit viral proteolytic processing during its late stage. Additionally, the protease inhibitors may have the advantages of being more readily available, longer lived in virus, and less toxic than currently available drugs, possibly due to their specificity for the retroviral protease.

On-going treatment of HIV-infected individuals with compounds that inhibit HIV protease has led to the development of mutant viruses that possess proteases that are resistant to the inhibitory effect of these compounds. Thus, to be effective, new HIV protease inhibitors must be effective not only against wild-type strains of HIV, but must also demonstrate efficacy against the newly emerging mutant strains that are resistant to the commercially available protease inhibitors. Accordingly, there continues to be a need for new inhibitors targeting the HIV protease in both wild type and mutant strains of HIV.

### SUMMARY

In one aspect of the present invention are provided methods for improving the pharmacokinetics of (4R)-N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide in a mammal, comprising administering to said mammal (4R)-N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide, or a pharmaceutically acceptable salt or solvate thereof, and at least one agent that inhibits the enzyme activity of cytochrome P450.

A method according to claim 1, wherein said (4R)-N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide, or a pharmaceutically acceptable salt or solvate thereof, and said at least one agent that inhibits the enzyme activity of cytochrome P450 are present in the same unitary dosage form.

In one aspect of the present invention are provided methods for improving the pharmacokinetics of (4R)-N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide in a mammal, comprising administering to said mammal a composition comprising (4R)-N-allyl-3-{(2S,3S)-2-hydroxy-3-[(3-hydroxy-2-methylbenzoyl)amino]-4-phenylbutanoyl}-5,5-dimethyl-1,3-thiazolidine-4carboxamide, or a pharmaceutically acceptable salt or solvate thereof, and at least one agent that inhibits the enzyme activity of cytochrome P450.

In one aspect of the present invention are provided methods for improving the pharmacokinetics of (4-N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide in a mammal, wherein said (4R)-N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide, or a pharmaceutically acceptable salt or solvate thereof, is administered to said mammal in an amount from about 100 mg to about 2000 mg.

In one aspect of the present invention are provided methods for improving the pharmacokinetics of 4,4-difluoro-1-{(2S,3S)-2-hydroxy-3-[(3-hydroxy-2-methylbenzoyl)amino]-4-phenylbutanoyl}-3,3-dimethyl-N-(2,2,2-trifluoroethyl)-L-prolinamide in a mammal, comprising administering to said mammal a composition comprising 4,4-difluoro-1-{(2S,3S)-2-hydroxy-3-[(3-hydroxy-2-methylbenzoyl)amino]-4-phenylbutanoyl}-3,3-dimethyl-N-(2,2,2-trifluoroethyl)-L-prolinamide, or a pharmaceutically acceptable salt or solvate thereof, and at least one agent that inhibits the enzyme activity of cytochrome P450.

In one aspect of the present invention are provided methods for improving the pharmacokinetics of 4,4-difluoro-1-{(2S,3S)-2-hydroxy-3-[(3-hydroxy-2-methylbenzoyl)amino]-4-phenylbutanoyl}-3,3-dimethyl-N-(2,2,2-trifluoroethyl)-L-prolinamide in a mammal, wherein said 4,4-difluoro-1-{(2S,3S)-2-hydroxy-3-[(3-hydroxy-2-methylbenzoyl)amino]-4-phenylbutanoyl}-3,3-dimethyl-N-(2,2,2-trifluoroethyl)-L-prolinamide, or a pharmaceutically acceptable salt or solvate thereof, is administered to said mammal in an amount from about 100 mg to about 2000 mg.

In one aspect of the present invention are provided methods for improving the pharmacokinetics of N-ethyl-4,4-difluoro-1-{(2S,3S)-2-hydroxy-3-[(3-hydroxy-2,5-dimethylbenzoyl)amino]-4-phenylbutanoyl}-3,3-dimethyl-L-prolinamide in a mammal, comprising administering to said mammal a composition comprising N-ethyl-4,4-difluoro-1-{(2S,3S)-2-hydroxy-3-[(3-hydroxy-2,5-dimethylbenzoyl)amino]-4-phenylbutanoyl}-3,3-dimethyl-L-prolinamide, or a pharmaceutically acceptable salt or solvate thereof, and at least one agent that inhibits the enzyme activity of cytochrome P450.

In one aspect of the present invention are provided methods for improving the pharmacokinetics of N-ethyl-4,4-difluoro-1-{(2S,3S)-2-hydroxy-3-[(3-hydroxy-2,5-dimethylbenzoyl)amino]-4-phenylbutanoyl}-3,3-dimethyl-L-prolinamide in a mammal, wherein said N-ethyl-4,4-difluoro-1-{(2S,3S)-2-hydroxy-3-[(3-hydroxy-2,5-dimethylbenzoyl)amino]-4-phenylbutanoyl}-3,3-dimethyl-L-prolinamide, or a pharmaceutically acceptable salt or solvate thereof, is administered to said mammal in an amount from about 100 mg to about 2000 mg.

In one aspect of the present invention are provided methods for improving the pharmacokinetics of N-ethyl-4,4-difluoro-1-{(2S,3S)-2-hydroxy-3-[(3-hydroxy-2,5dimethylbenzoyl)amino]-4-phenylbutanoyl}-3,3-dimethyl-L-prolinamide, 4,4-difluoro-1-{(2S,3S)-2-hydroxy-3-[(3-hydroxy-2-methylbenzoyl)amino]-4-phenylbutanoyl}-3,3-dimethyl-N-(2,2,2-trifluoroethyl)-L-prolinamide, or (4R)-N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide in a mammal, wherein said at least one agent that inhibits the enzyme activity of cytochrome P450 is chosen from ritonavir and delavirdine.

In one aspect of the present invention are provided methods for improving the pharmacokinetics of N-ethyl-4,4-difluoro-1-{(2S,3S)-2-hydroxy-3-[(3-hydroxy-2,5-dimethylbenzoyl)amino]-4-phenylbutanoyl}-3,3-dimethyl-L-prolinamide, 4,4-difluoro-1-{(2S,3S)-2-hydroxy-3-[(3-hydroxy-2-methylbenzoyl)amino]-4-phenylbutanoyl}-3,3-dimethyl-N-(2,2,2-trifluoroethyl)-L-prolinamide, or (4R)-N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide in a mammal, wherein said at least one agent that inhibits the enzyme activity of cytochrome P450 is ritonavir.

In one aspect of the present invention are provided methods for improving the pharmacokinetics of N-ethyl-4,4-difluoro-1-{(2S,3S)-2-hydroxy-3-[(3-hydroxy-2,5-dimethylbenzoyl)amino]-4-phenylbutanoyl}-3,3-dimethyl-L-prolinamide, 4,4-difluoro-1-{(2S,3S)-2-hydroxy-3-[(3-hydroxy-2-methylbenzoyl)amino]-4-phenylbutanoyl}-3,3-dimethyl-N-(2,2,2-trifluoroethyl)-L-prolinamide, or (4R)-N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide in a mammal, wherein said at least one agent that inhibits the enzyme activity of cytochrome P450 is ritonavir, and ritonavir is administered to said mammal in an amount from about 10 mg to about 500 mg.

In one aspect of the present invention are provided methods for improving the pharmacokinetics of N-ethyl-4,4-difluoro-1-{(2S,3S)-2-hydroxy-3-[(3-hydroxy-2,5-dimethylbenzoyl)amino]-4-phenylbutanoyl}-3,3-dimethyl-L-prolinamide, 4,4-difluoro-1-{(2S,3S)-2-hydroxy-3-[(3-hydroxy-2-methylbenzoyl)amino]-4-phenylbutanoyl}-3,3-dimethyl-N-(2,2,2-trifluoroethyl)-L-prolinamide, or (4R)-N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide in a mammal, wherein said at least one agent that inhibits the enzyme activity of cytochrome P450 is ritonavir, and ritonavir is administered to said mammal in an amount from about 50 mg to about 400 mg.

In one aspect of the present invention are provided methods for improving the pharmacokinetics of N-ethyl-4,4-difluoro-1-{(2S,3S)-2-hydroxy-3-[(3-hydroxy-2,5-dimethylbenzoyl)amino]-4-phenylbutanoyl}-3,3-dimethyl-L-prolinamide, 4,4-difluoro-1-{(2S,3S)-2-hydroxy-3-[(3-hydroxy-2-methylbenzoyl)amino]-4-phenylbutanoyl}-3,3-dimethyl-N-(2,2,2trifluoroethyl)-L-prolinamide, or (4R)-N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide in a mammal, wherein said at least one agent that inhibits the enzyme activity of cytochrome P450 is ritonavir, and ritonavir is administered to said mammal in an amount from about 100 mg to about 200 mg.

In one aspect of the present invention are provided methods for improving the pharmacokinetics of N-ethyl-4,4-difluoro-1-{(2S,3S)-2-hydroxy-3-[(3-hydroxy-2,5-dimethylbenzoyl)amino]-4-phenylbutanoyl}-3,3-dimethyl-L-prolinamide, 4,4-difluoro-1-{(2S,3S)-2-hydroxy-3-[(3-hydroxy-2-methylbenzoyl)amino]-4-phenylbutanoyl}-3,3-dimethyl-N-(2,2,2-trifluoroethyl)-L-prolinamide, or (4R)-N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide in a mammal, wherein said at least one agent that inhibits the enzyme activity of cytochrome P450 is delavirdine.

In one aspect of the present invention are provided methods for improving the pharmacokinetics of N-ethyl-4,4-difluoro-1-{(2S,3S)-2-hydroxy-3-[(3-hydroxy-2,5-dimethylbenzoyl)amino]-4-phenylbutanoyl}-3,3-dimethyl-L-prolinamide, 4,4-difluoro-1-{(2S,3S)-2-hydroxy-3-[(3-hydroxy-2-methylbenzoyl)amino]-4-phenylbutanoyl}-3,3-dimethyl-N-(2,2,2-trifluoroethyl)-L-prolinamide, or (4R)-N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide in a mammal, wherein said at least one agent that inhibits the enzyme activity of cytochrome P450 is delavirdine and is administered to said mammal in an amount from about 10 mg to about 500 mg.

In one aspect of the present invention are provided methods for improving the pharmacokinetics of N-ethyl-4,4-difluoro-1 {(2S,3S)-2-hydroxy-3-[(3-hydroxy-2,5-dimethylbenzoyl)amino]-4-phenylbutanoyl]-3,3-dimethyl-L-prolinamide, 4,4-difluoro-1-{(2S,3S)-2-hydroxy-3-[(3-hydroxy-2-methylbenzoyl)amino]-4-phenylbutanoyl}-3,3-dimethyl-N-(2,2,2-trifluoroethyl)-L-prolinamide, or (4R)-N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide in a mammal, wherein said at least one agent that inhibits the enzyme activity of cytochrome P450 is delavirdine and is administered to said mammal in an amount from about 50 mg to about 400 mg.

In one aspect of the present invention are provided methods for improving the pharmacokinetics of N-ethyl-4,4-difluoro-1-{(2S,3S)-2-hydroxy-3-[(3-hydroxy-2,5-dimethylbenzoyl)amino]-4-phenylbutanoyl}-3,3-dimethyl-L-prolinamide, 4,4-difluoro-1-{(2S,3S)-2-hydroxy-3-[(3-hydroxy-2-methylbenzoyl)amino]-4-phenylbutanoyl}-3,3-dimethyl-N-(2,2,2-trifluoroethyl)-L-prolinamide, or (4R)-N-allyl-3-{(2S,3S)-2-hydroxy-3-[(3-hydroxy-2methylbenzoyl)amino]-4-phenylbutanoyl}-5,5-dimethyl-1,3-thiazolidine-4-carboxamide in a mammal, wherein said at least one agent that inhibits the enzyme activity of cytochrome P450 is delavirdine and is administered to said mammal in an amount from about 100 mg to about 200 mg.

In one aspect of the present invention are provided methods according to any one the methods described above, wherein said administration is sequential.

In one aspect of the present invention are provided methods according to any one the methods described above, wherein said administration is simultaneous.

In one aspect of the present invention are provided methods according to any one the methods described above, wherein said administration is performed once a day.

In one aspect of the present invention are provided methods according to any one the methods described above, wherein said administration is performed twice a day.

In one aspect of the present invention are provided methods according to any one the methods described above, wherein said administration is performed three times a day.

In one aspect of the present invention are provided methods as described above, wherein said (4R)-N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide, or a pharmaceutically acceptable salt or solvate thereof, and at least one agent that inhibits the enzyme activity of cytochrome P450, are present in an amount sufficient that (4R)-N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide is present in the plasma of said mammal in a concentration of from about 0.001 μM to about 10 μM for at least about 6 hours after said administration.

In one aspect of the present invention are provided methods as described above, wherein said plasma concentration of said (4R)-N-allyl-3-{(2S,3S)-2-hydroxy-3-[(3-hydroxy-R-methylbenzoyl)amino]-4-phenylbutanoyl}-5,5-dimethyl-1,3-thiazolidine-4-carboxamide is from about 0.01 μM to about 5 μM for at least about 6 hours after said administration.

In one aspect of the present invention are provided methods as described above, wherein said plasma concentration of said (4R)-N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide is from about 0.01 μM to about 2.5 μM for at least about 6 hours after said administration.

In one aspect of the present invention are provided methods as described above, wherein said plasma concentration of said (4R)-N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide is from about 0.01 μM to about 2.5 μM for at least about 6 hours after said administration.

In one aspect of the present invention are provided methods as described above, wherein said plasma concentration of said (4R)-N-allyl-3-{(2S,3S)-2-hydroxy-3-[(3-hydroxy-2methylbenzoyl)amino]-4-phenylbutanoyl}-5,5-dimethyl-1,3-thiazolidine-4-carboxamide is from about 0.02 μM to about 1 μM for at least about 6 hours after said administration.

In one aspect of the present invention are provided methods as described above, wherein said plasma concentration of said (4R)-N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide is from about 0.025 μM to about 1 μM for at least about 6 hours after said administration.

In one aspect of the present invention are provided methods as described above, wherein said 4,4-difluoro-1-{(2S,3S)-2-hydroxy-3-[(3-hydroxy-2-methylbenzoyl)amino]-4-phenylbutanoyl}-3,3-dimethyl-N-(2,2,2-trifluoroethyl)-L-prolinamide, or a pharmaceutically acceptable salt or solvate thereof, and at least one agent that inhibits the enzyme activity of cytochrome P450, are present in an amount sufficient that 4,4-difluoro-1-{(2S,3S)-2-hydroxy-3-[(3-hydroxy-2-methylbenzoyl)amino]-4-phenylbutanoyl}-3,3-dimethyl-N-(2,2,2-trifluoroethyl)-L-prolinamide is present in the plasma of said mammal in a concentration of from about 0.001 μM to about 10 μM for at least about 6 hours after said administration.

In one aspect of the present invention are provided methods as described above, wherein said plasma concentration of said 4,4-difluoro-1-{(2S,3S)-2-hydroxy-3-[(3-hydroxy-2-methylbenzoyl)amino]-4-phenylbutanoyl}-3,3-dimethyl-N-(2,2,2-trifluoroethyl)-L-prolinamide is from about 0.01 μM to about 5 μM for at least about 6 hours after said administration.

In one aspect of the present invention are provided methods as described above, wherein said plasma concentration of said (4,4-difluoro-1-{(2S,3S)-2-hydroxy-3-[(3-hydroxy-2-methylbenzoyl)amino]-4-phenylbutanoyl}-3,3-dimethyl-N-(2,2,2-trifluoroethyl)-L-prolinamide is from about 0.01 μM to about 2.5 μM for at least about 6 hours after said administration.

In one aspect of the present invention are provided methods as described above, wherein said plasma concentration of said (4,4-difluoro-1-{(2S,3S)-2-hydroxy-3-[(3-hydroxy-2-methylbenzoyl)amino]-4-phenylbutanoyl}-3,3-dimethyl-N-(2,2,2-trifluoroethyl)-L-prolinamide is from about 0.01 μM to about 2.5 μM for at least about 6 hours after said administration.

In one aspect of the present invention are provided methods as described above, wherein said plasma concentration of said 4,4-difluoro-1-{(2S,3S)-2-hydroxy-3-[(3-hydroxy-2-methylbenzoyl)amino]-4-phenylbutanoyl}-3,3-dimethyl-N-(2,2,2-trifluoroethyl)-L-prolinamide is from about 0.02 μM to about 1 μM for at least about 6 hours after said administration.

In one aspect of the present invention are provided methods as described above, wherein said plasma concentration of said 4,4-difluoro-1-{(2S,3S)-2-hydroxy-3-[(3-hydroxy-2-methylbenzoyl)amino]-4-phenylbutanoyl}-3,3-dimethyl-N-(2,2,2-trifluoroethyl)-L-prolinamide is from about 0.025 μM to about 1 μM for at least about 6 hours after said administration.

In one aspect of the present invention are provided methods as described above, wherein said N-ethyl-4,4-difluoro-1-{(2S,3S)-2-hydroxy-3-[(3-hydroxy-2,5dimethylbenzoyl)amino]-4-phenylbutanoyl}-3,3-dimethyl-L-prolinamide, or a pharmaceutically acceptable salt or solvate thereof, and at least one agent that inhibits the enzyme activity of cytochrome P450, are present in an amount sufficient that N-ethyl-4,4-difluoro-1-{(2S,3S)-2-hydroxy-3-[(3-hydroxy-2,5-dimethylbenzoyl)amino]-4-phenylbutanoyl}-3,3-dimethyl-L-prolinamide is present in the plasma of said mammal in a concentration of from about 0.001 μM to about 10 μM for at least about 6 hours after said administration.

In one aspect of the present invention are provided methods as described above, wherein said plasma concentration of said N-ethyl-4,4-difluoro-1-{(2S,3S)-2-hydroxy-3-[(3-hydroxy-2,5-dimethylbenzoyl)amino]-4-phenylbutanoyl}-3,3-dimethyl-L-prolinamide is from about 0.01 μM to about 5 μM for at least about 6 hours after said administration.

In one aspect of the present invention are provided methods as described above, wherein said plasma concentration of said N-ethyl-4,4-difluoro-1-{(2S,3S)-2-hydroxy-3-[(3-hydroxy-2,5-dimethylbenzoyl)amino]-4-phenylbutanoyl}-3,3-dimethyl-L-prolinamide is from about 0.01 μM to about 2.5 μM for at least about 6 hours after said administration.

In one aspect of the present invention are provided methods as described above, wherein said plasma concentration of said N-ethyl-4,4-difluoro-1-{(2S,3S)-2-hydroxy-3-[(3-hydroxy-2,5-dimethylbenzoyl)amino]-4-phenylbutanoyl}-3,3-dimethyl-L-prolinamide is from about 0.01 μM to about 2.5 μM for at least about 6 hours after said administration.

In one aspect of the present invention are provided methods as described above, wherein said plasma concentration of said N-ethyl-4,4-difluoro-1-{(2S,3S)-2-hydroxy-3-[(3-hydroxy-2,5-dimethylbenzoyl)amino]-4-phenylbutanoyl}-3,3-dimethyl-L-prolinamide is from about 0.02 μM to about 1 μM for at least about 6 hours after said administration.

In one aspect of the present invention are provided methods as described above, wherein said plasma concentration of said 4,4-difluoro-1-{(2S,3S)-2-hydroxy-3-[(3-hydroxy-2-methylbenzoyl)amino]-4-phenylbutanoyl}-3,3-dimethyl-N-(2,2,2-trifluoroethyl)-L-prolinamide is from about 0.025 μM to about 1 μM for at least about 6 hours after said administration.

In one aspect of the present invention are provided methods as described above, wherein said (4R)-N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide, or a pharmaceutically acceptable salt or solvate thereof, and said at least one agent that inhibits the enzyme activity of cytochrome P450, are present in an amount sufficient that the average plasma concentration of said (4R)-N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide in said mammal is in the range of from about 0.001 μM to about 10 μM for about 6 to about 24 hours following said administration.

In one aspect of the present invention are provided methods as described above, wherein said average plasma concentration of (4R)-N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide in said mammal is in the range of from about 0.01 μM to about 2.5 μM for about 6 to about 24 hours following said administration.

In one aspect of the present invention are provided methods as described above, wherein said average plasma concentration of (4R)-N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide in said mammal is in the range of from about 0.01 μM to about 2.0 μM for about 6 to about 24 hours following said administration.

In one aspect of the present invention are provided methods as described above, wherein said average plasma concentration of (4R)-N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide in said mammal is in the range of from about 0.01 μM to about 1.5 μM for about 6 to about 24 hours following said administration.

In one aspect of the present invention are provided methods as described above, wherein said average plasma concentration of (4R)-N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide in said mammal is in the range of from about 0.02 μM to about 1 μM for about 6 to about 24 hours following said administration.

In one aspect of the present invention are provided methods as described above, wherein said 4,4-difluoro-1-{(2S,3S)-2-hydroxy-3-[(3-hydroxy-2-methylbenzoyl)amino]-4-phenylbutanoyl}-3,3-dimethyl-N-(2,2,2-trifluoroethyl)-L-prolinamide, or a pharmaceutically acceptable salt or solvate thereof, and said at least one agent that inhibits the enzyme activity of cytochrome P450, are present in an amount sufficient that the average plasma concentration of said 4,4-difluoro-1-{(2S,3S)-2-hydroxy-3-[(3-hydroxy-2-methylbenzoyl)amino]-4-phenylbutanoyl}-3,3-dimethyl-N-(2,2,2-trifluoroethyl)-L-prolinamide in said mammal is in the range of from about 0.001 μM to about 10 μM for about 6 to about 24 hours following said administration.

In one aspect of the present invention are provided methods as described above, wherein said average plasma concentration of 4,4-difluoro-1-{(2S,3S)-2-hydroxy-3-[(3-hydroxy-2-methylbenzoyl)amino]-4-phenylbutanoyl}-3,3-dimethyl-N-(2,2,2-trifluoroethyl)-L-prolinamide in said mammal is in the range of from about 0.01 μM to about 2.5 μM for about 6 to about 24 hours following said administration.

In one aspect of the present invention are provided methods according as described above, wherein said average plasma concentration of 4,4-difluoro-1-{(2S,3S)-2-hydroxy-3-[(3-hydroxy-2-methylbenzoyl)amino]-4-phenylbutanoyl}-3,3-dimethyl-N-(2,2,2-trifluoroethyl)-L-prolinamide in said mammal is in the range of from about 0.01 μM to about 2.0 μM for about 6 to about 24 hours following said administration.

In one aspect of the present invention are provided methods as described above, wherein said average plasma concentration of 4,4-difluoro-1-{(2S,3S)-2-hydroxy-3-[(3-hydroxy-2-methylbenzoyl)amino]-4-phenylbutanoyl}-3,3-dimethyl-N-(2,2,2-trifluoroethyl)-L-prolinamide in said mammal is in the range of from about 0.01 μM to about 1.5 μM for about 6 to about 24 hours following said administration.

In one aspect of the present invention are provided methods as described above, wherein said average plasma concentration of 4,4-difluoro-1-{(2S,3S)-2-hydroxy-3-[(3-hydroxy-2-methylbenzoyl)amino]-4-phenylbutanoyl}-3,3-dimethyl-N-(2,2,2-trifluoroethyl)-L-prolinamide in said mammal is in the range of from about 0.02 μM to about 1 μM for about 6 to about 24 hours following said administration.

In one aspect of the present invention are provided methods as described above, wherein said N-ethyl-4,4-difluoro-1-{(2S,3S)-2-hydroxy-3-[(3-hydroxy-2,5-dimethylbenzoyl)amino]-4-phenylbutanoyl}-3,3-dimethyl-L-prolinamide, or a pharmaceutically acceptable salt or solvate thereof, and said at least one agent that inhibits the enzyme activity of cytochrome P450, are present in an amount sufficient that the average plasma concentration of said N-ethyl-4,4-difluoro-1-{(2S,3S)-2-hydroxy-3-[(3-hydroxy-2,5-dimethylbenzoyl)amino]-4-phenylbutanoyl}-3,3-dimethyl-L-prolinamide in said mammal is in the range of from about 0.001 μM to about 10 μM for about 6 to about 24 hours following said administration.

In one aspect of the present invention are provided methods as described above, wherein said average plasma concentration of N-ethyl-4,4-difluoro-1-{(2S,3S)-2-hydroxy-3-[(3-hydroxy-2,5-dimethylbenzoyl)amino]-4-phenylbutanoyl}-3,3-dimethyl-L-prolinamide in said mammal is in the range of from about 0.01 μM to about 2.5 μM for about 6 to about 24 hours following said administration.

In one aspect of the present invention are provided methods as described above, wherein said average plasma concentration of N-ethyl-4,4-difluoro-1-{(2S,3S)-2-hydroxy-3-[(3-hydroxy-2,5-dimethylbenzoyl)amino]-4-phenylbutanoyl}-3,3-dimethyl-L-prolinamide in said mammal is in the range of from about 0.01 μM to about 2.0 μM for about 6 to about 24 hours following said administration.

In one aspect of the present invention are provided methods as described above, wherein said average plasma concentration of N-ethyl-4,4-difluoro-1-{(2S,3S)-2-hydroxy-3-[(3-hydroxy-2,5-dimethylbenzoyl)amino]-4-phenylbutanoyl}-3,3-dimethyl-L-prolinamide in said mammal is in the range of from about 0.01 μM to about 1.5 μM for about 6 to about 24 hours following said administration.

In one aspect of the present invention are provided methods as described above, wherein said average plasma concentration of N-ethyl-4,4-difluoro-1-{(2S,3S)-2-hydroxy-3-[(3-hydroxy-2,5-dimethylbenzoyl)amino]-4-phenylbutanoyl}-3,3-dimethyl-L-prolinamide in said mammal is in the range of from about 0.02 μM to about 1 μM for about 6 to about 24 hours following said administration.

In one aspect of the present invention are provided methods as described above, wherein said at least one agent that inhibits the enzyme activity of cytochrome P450 is chosen from ritonavir or delavirdine.

In one aspect of the present invention are provided methods as described above, wherein said at least one agent that inhibits the enzyme activity of cytochrome P450 is ritonavir.

In one aspect of the present invention are provided methods as described above, wherein said at least one agent that inhibits the enzyme activity of cytochrome P450 is delavirdine.

In one aspect of the present invention are provided methods as described above, wherein said cytochrome P450 is the 3A4 isoform.

In one aspect of the present invention are provided compositions comprising (4R)-N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide, or a pharmaceutically acceptable salt or solvate thereof, and at least one agent that inhibits the enzyme activity of cytochrome P450.

In one aspect of the present invention are provided compositions as described above, wherein said at least one agent that inhibits the enzyme activity of cytochrome P450 is chosen from ritonavir and delavirdine.

In one aspect of the present invention are provided compositions as described above, wherein said at least one agent that inhibits the enzyme activity of cytochrome P450 is ritonavir.

In one aspect of the present invention are provided compositions as described above, wherein said at least one agent that inhibits the enzyme activity of cytochrome P450 is delavirdine.

In one aspect of the present invention are provided compositions as described above, wherein said cytochrome P450 is the 3A4 isoform.

In one aspect of the present invention are provided compositions as described above, wherein said (4R)-N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide, or a pharmaceutically acceptable salt or solvate thereof, and ritonavir are present in a ratio of from about 1:1 to about 20:1 by weight.

In one aspect of the present invention are provided compositions as described above, wherein said (4R)-N-allyl-3-{(2S,3S)-2-hydroxy-3-[(3-hydroxy-2-methylbenzoyl)amino]-4phenylbutanoyl}-5,5-dimethyl-1,3-thiazolidine-4-carboxamide, or a pharmaceutically acceptable salt or solvate thereof, and ritonavir are present in a ratio of from about 1:1 to about 10:1 by weight.

In one aspect of the present invention are provided compositions as described above, wherein said (4R)-N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide, or a pharmaceutically acceptable salt or solvate thereof, and ritonavir are present in a molar ratio of from about 1:0.1 to about 10:1.

In one aspect of the present invention are provided compositions comprising 4,4-difluoro-1-{(2S,3S)-2-hydroxy-3-[(3-hydroxy-2-methylbenzoyl)amino]-4-phenylbutanoyl}-3,3-dimethyl-N-(2,2,2-trifluoroethyl)-L-prolinamide, or a pharmaceutically acceptable salt or solvate thereof, and at least one agent that inhibits the enzyme activity of cytochrome P450.

In one aspect of the present invention are provided compositions as described above, wherein said at least one agent that inhibits the enzyme activity of cytochrome P450 is chosen from ritonavir and delavirdine.

In one aspect of the present invention are provided compositions as described above, wherein said at least one agent that inhibits the enzyme activity of cytochrome P450 is ritonavir.

In one aspect of the present invention are provided compositions as described above wherein said at least one agent that inhibits the enzyme activity of cytochrome P450 is delavirdine.

In one aspect of the present invention are provided compositions as described above, wherein said cytochrome P450 is the 3A4 isoform.

In one aspect of the present invention are provided compositions as described above, wherein said 4,4-difluoro-1-{(2S,3S)-2-hydroxy-3-[(3-hydroxy-2-methylbenzoyl)amino]-4-phenylbutanoyl}-3,3-dimethyl-N-(2,2,2-trifluoroethyl)-L-prolinamide, or a pharmaceutically acceptable salt or solvate thereof, and ritonavir are present in a ratio of from about 1:1 to about 20:1 by weight.

In one aspect of the present invention are provided compositions as described above, wherein said 4,4-difluoro-1-{(2S,3S)-2-hydroxy-3-[(3-hydroxy-2-methylbenzoyl)amino]-4-phenylbutanoyl}-3,3-dimethyl-N-(2,2,2-trifluoroethyl)-L-prolinamide, or a pharmaceutically acceptable salt or solvate thereof, and ritonavir are present in a ratio of from about 1:1 to about 10:1 by weight.

In one aspect of the present invention are provided compositions as described above, wherein said 4,4-difluoro-1-{(2S,3S)-2-hydroxy-3-[(3-hydroxy-2-methylbenzoyl)amino]-4-phenylbutanoyl}-3,3-dimethyl-N-(2,2,2-trifluoroethyl)-L-prolinamide, or a pharmaceutically acceptable salt or solvate thereof, and ritonavir are present in a molar ratio of from about 1:0.1 to about 10:1.

In one aspect of the present invention are provided compositions comprising N-ethyl-4,4-difluoro-1{(2S,3S)-2-hydroxy-3-[(3-hydroxy-2,5-dimethylbenzoyl)amino]-4-phenylbutanoyl}-3,3-dimethyl-L-prolinamide, or a pharmaceutically acceptable salt or solvate thereof, and at least one agent that inhibits the enzyme activity of cytochrome P450.

In one aspect of the present invention are provided compositions as described above, wherein said at least one agent that inhibits the enzyme activity of cytochrome P450 is chosen from ritonavir and delavirdine.

In one aspect of the present invention are provided compositions as described above, wherein said at least one agent that inhibits the enzyme activity of cytochrome P450 is ritonavir.

In one aspect of the present invention are provided compositions as described above, wherein said at least one agent that inhibits the enzyme activity of cytochrome P450 is delavirdine.

In one aspect of the present invention are provided compositions as described above, wherein said cytochrome P450 is the 3A4 isoform.

In one aspect of the present invention are provided compositions as described above, wherein said (4R)-N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide, or a pharmaceutically acceptable salt or solvate thereof, and ritonavir are present in a ratio of from about 1:1 to about 20:1 by weight.

In one aspect of the present invention are provided compositions as described above, wherein said (4R)-N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide, or a pharmaceutically acceptable salt or solvate thereof, and ritonavir are present in a ratio of from about 1:1 to about 10:1 by weight.

In one aspect of the present invention are provided compositions as described above, wherein said (4R)-N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide, or a pharmaceutically acceptable salt or solvate thereof, and ritonavir are present in a molar ratio of from about 1:0.1 to about 10:1.

The terms “cytochrome P450-inhibiting amount” and “cytochrome P450 enzyme activity-inhibiting amount,” as used herein, refer to an amount of a compound required to decrease the activity of cytochrome P450 enzymes or a particular cytochrome P450 enzyme isoform in the presence of such compound. Whether a particular compound of decreases cytochrome P450 enzyme activity, and the amount of such a compound required to do so, can be determined by methods known to those of ordinary skill in the art and the methods described herein.

The terms “inhibiting” or “inhibition,” as used herein, refer to decreasing the activity of a cytochrome P450 enzyme or enzymes using an agent that is capable of decreasing such activity either in vitro or in vivo after administration to a mammal, such as a human. Such inhibition may take place by the compound binding directly to the cytochrome P450 enzyme or enzymes. In addition, the activity of such cytochrome P450 enzymes may be decreased in the presence of such a compound when such direct binding between the enzyme and the compound does not take place. Furthermore, such inhibition may be competitive, non-competitive, or uncompetitive, as described in T. F. Woolf, Handbook of Drug Metabolism, Marcel Dekker, Inc., New York, 1999. Such inhibition may be determined using in vitro or in vivo systems, or a combination of both, using methods known to those of ordinary skill in the art.

As used herein, the term “bioavailability” refers to the systemic availability of a given amount of a chemical compound administered to a mammal. Bioavailability can be assessed by measuring the area under the curve (AUC) or the maximum serum or plasma concentration (Cₘₐₓ) of the unchanged form of a compound following administration of the compound to a mammal. AUC is a determination of the Area Under the Curve plotting the serum or plasma concentration of a compound along the ordinate (Y-axis) against time along the abscissa α-axis). Generally, the AUC for a particular compound can be calculated using methods known to those of ordinary skill in the art and as described in G. S. Banker, Modern Pharmaceutics, Drugs and the Pharmaceutical Sciences, V. 72, Marcel Dekker, New York, Inc., 1996. The Cₘₐₓ value is defined as the maximum concentration of the compound achieved in the serum or plasma of a mammal following administration of the compound to the mammal. The Cₘₐₓ value of a particular compound can be measured using methods known to those of ordinary skill in the art. The phrase “increasing bioavailability,” as used herein means that the systemic availability of a first compound, measured as AUC or Cₘₐₓ, in a mammal is greater when co-administered with a compound of the present invention than when such co-administration does not take place.

The terms “administration”, “administering”, “dosage,” and “dosing,” as used herein refer to the delivery of a compound, or a pharmaceutically acceptable salt or solvate thereof, or of a pharmaceutical composition containing the compound, or a pharmaceutically acceptable salt or solvate thereof, to a mammal such that the compound is absorbed into the serum or plasma of the mammal.

The terms “co-administration” or “co-administering,” as used herein, refer to the administration of a combination of a first compound and a compound of the present invention, or a pharmaceutically acceptable salt or solvate thereof. Such co-administration can be performed such that the first compound and the compound of the present invention are part of the same composition or part of the same unitary dosage form. Co-administration also includes administering a first compound and a compound of the present invention separately, but as part of the same therapeutic regimen. The two components, if administered separately, need not necessarily be administered at essentially the same time, although they can be if so desired. Thus co-administration includes, for example, administering a first compound and a compound of the present invention as separate dosages or dosage forms, but at the same time. Co-administration also includes separate administration at different times and in any order.

The phrase “pharmaceutically acceptable salt(s)”, as used herein, unless otherwise indicated, includes salts of acidic or basic groups, which may be present in the compounds of the present invention.

The term “solvate,” as used herein, is intended to mean a compound of the present invention in a form such that a molecule of solvent is associated with a molecule of the present invention. It is specifically contemplated that in the present invention one solvent molecule can be associated with one molecule of the present invention, such as a hydrate. Furthermore, it is specifically contemplated that in the present invention, more than one solvent molecule may be associated with one molecule of the present invention, such as a dihydrate. Additionally, it is specifically contemplated that in the present invention less than one solvent molecule may be associated with one molecule of the present invention, such as a hemihydrate. Furthermore, solvates of the present invention are contemplated as solvates of compounds of the present invention that retain the biological effectiveness of the non-hydrate form of the compounds.

### DETAILED DESCRIPTION

A “solvate” is intended to mean a pharmaceutically acceptable solvate form of a specified compound that retains the biological effectiveness of such compound. Examples of solvates include, but are not limited to, compounds of the invention in combination with water, isopropanol, ethanol, methanol, dimethylsulfoxide (DMSO), ethyl acetate, acetic acid, ethanolamine, or mixtures thereof.

A “pharmaceutically acceptable salt” is intended to mean a salt that retains the biological effectiveness of the free acids and bases of the specified derivative, containing pharmacologically acceptable anions, and is not biologically or otherwise undesirable. Examples of pharmaceutically acceptable salts include, but are not limited to, acetate, acrylate, benzenesulfonate, benzoate (such as chlorobenzoate, methylbenzoate, dinitrobenzoate, hydroxybenzoate, and methoxybenzoate), bicarbonate, bisulfate, bisulfite, bitartrate, borate, bromide, butyne-1,4-dioate, calcium edetate, camsylate, carbonate, chloride, caproate, caprylate, clavulanate, citrate, decanoate, dihydrochloride, dihydrogenphosphate, edetate, edislyate, estolate, esylate, ethylsuccinate, formate, fumarate, gluceptate, gluconate, glutamate, glycollate, glycollylarsanilate, heptanoate, hexyne-1,6-dioate, hexylresorcinate, hydrabamine, hydrobromide, hydrochloride, γ-hydroxybutyrate, iodide, isobutyrate, isothionate, lactate, lactobionate, laurate, malate, maleate, malonate, mandelate, mesylate, metaphosphate, methane-sulfonate, methylsulfate, monohydrogenphosphate, mucate, napsylate, naphthalene-1-sulfonate, naphthalene-2-sulfonate, nitrate, oleate, oxalate, pamoate (embonate), palmitate, pantothenate, phenylacetates, phenylbutyrate, phenylpropionate, phthalate, phospate/diphosphate, polygalacturonate, propanesulfonate, propionate, propiolate, pyrophosphate, pyrosulfate, salicylate, stearate, subacetate, suberate, succinate, sulfate, sulfonate, sulfite, tannate, tartrate, teoclate, tosylate, triethiodode, and valerate salts.

It is understood by those of ordinary skill in the art that the compounds of the present invention, or their pharmaceutically acceptable salts or solvates, may exist in different polymorph or crystal forms, all of which are intended to be within the scope of the present invention and specified formulas. In addition, the compounds of the present invention, and their pharmaceutically acceptable salts and solvates, may exist as tautomers, all of which are intended to be within the broad scope of the present invention.

Administration of the compounds, or their pharmaceutically acceptable salts or solvates, may be performed according to any suitable mode of administration available to one of ordinary skill in the art. Examples of such suitable modes of administration include oral, nasal, parenteral, topical, transdermal, rectal, or by inhalation or spray.

For example, such delivery may be performed by orally administering a first compound, or a pharmaceutically acceptable salt thereof, and a compound of the invention, or a pharmaceutically acceptable salt thereof, to a mammal, such as a human. Furthermore, the first compound and a compound of the present invention, and any additional compounds, may be administered in the form of a pharmaceutically acceptable formulation containing non-toxic, pharmaceutically acceptable carriers, adjuvants and vehicles. Alternatively, the first compound and a compound of formula (I), or pharmaceutically acceptable salts or solvates thereof, may be administered to a mammal by other routes of administration including, but not limited to, intravenous, topical, sublingual, parenteral, rectal, or by inhalation or spray. Such alternative administration may be performed with the first compound and a compound of the present invention alone or in dosage unit formulations containing non-toxic, pharmaceutically acceptable carriers, adjuvants and vehicles. In addition, the present invention specifically contemplates that the first compound and the compound of the present invention may be co-administered using different forms of administration for each. For example, the first compound may be administered topically while the compound of formula (I), or a pharmaceutically acceptable salt or solvate thereof, may be administered orally. The preferred formulation and route of administration of the first compound and the compound of the present invention to a mammal will depend on the age and condition of the mammal, the condition being treated, the identity of the first compound, the identity of the compound of the present invention, and other factors known to those of ordinary skill in the art. The formulation and route of administration can be determined by one of ordinary skill in the art without undue experimentation.

Acceptable methods of preparing suitable pharmaceutical forms of the pharmaceutical compositions are known or may be routinely determined by those skilled in the art. For example, pharmaceutical preparations may be prepared following conventional techniques of the pharmaceutical chemist involving steps such as mixing, granulating, and compressing when necessary for tablet forms, or mixing, filling, and dissolving the ingredients as appropriate, to give the desired products for oral, parenteral, topical, intravaginal, intranasal, intrabronchial, intraocular, intraaural, and/or rectal administration.

Pharmaceutical compositions of the invention may also include suitable excipients, diluents, vehicles, and carriers, as well as other pharmaceutically active agents, depending upon the intended use. Solid or liquid pharmaceutically acceptable carriers, diluents, vehicles, or excipients may be employed in the pharmaceutical compositions. Illustrative solid carriers include starch, lactose, calcium sulfate dihydrate, terra alba, sucrose, talc, gelatin, pectin, acacia, magnesium stearate, and stearic acid. Illustrative liquid carriers include syrup, peanut oil, olive oil, saline solution, and water. The carrier or diluent may include a suitable prolonged-release material, such as glyceryl monostearate or glyceryl distearate, alone or with a wax. When a liquid carrier is used, the preparation may be in the form of a syrup, elixir, emulsion, soft gelatin capsule, sterile injectable liquid (e.g., solution), or a nonaqueous or aqueous liquid suspension.

Methods of preparing various pharmaceutical compositions with a specific amount of active compound are known, or will be apparent, to those skilled in this art. For examples, see Remington's Pharmaceutical Sciences, Mack Publishing Company, Easter, Pa., 15^{th} Edition (1975).

It will be appreciated that the actual dosages of the compounds of the present invention, or pharmaceutically acceptable salts thereof, used in the pharmaceutical compositions of this invention will be selected according to the properties of the particular agent being used, the particular composition formulated, the mode of administration, the particular site, the host, and the condition being treated. Optimal dosages for a given set of conditions can be ascertained by those skilled in the art using conventional dosage-determination tests. For oral administration, e.g., a dose that may be employed is from about 0.001 to about 1000 mg/kg body weight, preferably from about 0.1 to about 100 mg/kg body weight, and even more preferably from about 1 to about 50 mg/kg body weight, with courses of treatment repeated at appropriate intervals.

The amount and timing of compounds administered will, of course, be based on the judgment of the prescribing physician. Thus, because of subject-to-subject variability, the dosages provided are a guideline and one of ordinary skill in the art may titrate doses of the agent to achieve the activity that they consider appropriate for the individual subject. In considering the degree of activity desired, one of skill in the art must balance a variety of factors such as cognitive function, age of the patient, presence of preexisting disease, as well as presence of other diseases (e.g., cardiovascular).

The compositions of the present invention are generally administered in the form of a pharmaceutical composition comprising at least one of the compounds of this invention together with a pharmaceutically acceptable vehicle or diluent. Thus, the compounds of this invention can be administered individually or together with the first compound(s) in any conventional dosage form.

For oral administration a pharmaceutical composition can take the form of solutions, suspensions, tablets, pills, capsules, powders, and the like. Tablets containing various excipients such as sodium citrate, calcium carbonate and calcium phosphate are employed along with various disintegrants such as starch and preferably potato or tapioca starch and certain complex silicates, together with binding agents such as polyvinylpyrrolidone, sucrose, gelatin and acacia. Additionally, lubricating agents such as magnesium stearate, sodium lauryl sulfate and talc are often very useful for tabletting purposes. Solid compositions of a similar type are also employed as fillers in soft and hard-filled gelatin capsules; preferred materials in this connection also include lactose or milk sugar as well as high molecular weight polyethylene glycols. When aqueous suspensions and/or elixirs are desired for oral administration, the compounds of this invention can be combined with various sweetening agents, flavoring agents, coloring agents, emulsifying agents and/or suspending agents, as well as such diluents as water, ethanol, propylene glycol, glycerin and various like combinations thereof.

For purposes of parenteral administration, solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes. In this connection, the sterile aqueous media employed are all readily obtainable by standard techniques known to those skilled in the art.

For purposes of transdermal (e.g., topical) administration, dilute sterile, aqueous or partially aqueous solutions (usually in about 0.1% to 5% concentration), otherwise similar to the above parenteral solutions, are prepared.

Methods of preparing various pharmaceutical compositions with a certain amount of active ingredient are known, or will be apparent in light of this disclosure, to those skilled in this art. For examples, see Remington's Pharmaceutical Sciences, Mack Publishing Company, Easter, Pa., 15th Edition (1975).

The compounds of the present invention may be administered in combination with an additional agent or agents for the treatment of a mammal, such as a human, that is suffering from an infection with the HIV virus, AIDS, AIDS-related complex (ARC), or any other disease or condition which is related to infection with the HIV virus. The agents that may be used in combination with the compounds of the present invention include, but are not limited to, those useful as HIV protease inhibitors, HIV reverse transcriptase inhibitors, non-nucleoside HIV reverse transcriptase inhibitors, inhibitors of HIV integrase, CCR5 inhibitors, HIV fusion inhibitors, compounds useful as immunomodulators, compounds that inhibit the HIV virus by an unknown mechanism, compounds useful for the treatment of herpes viruses, compounds useful as anti-infectives, and others as described below.

Compounds useful as HIV protease inhibitors that may be used in combination with the compounds of the present invention include, but are not limited to, 141 W94 (amprenavir), CGP-73547, CGP-61755, DMP-450, nelfinavir, saquinavir (invirase), TMC-126, atazanavir, palinavir, GS-3333, KN I-413, KNI-272, LG-71350, CGP-61755, PD 173606, PD 177298, PD 178390, PD 178392, U-140690, ABT-378, DMP-450, AG-1776, MK-944, VX-478, indinavir, tipranavir, TMC-114, DPC-681, DPC-684, fosamprenavir calcium (Lexiva), benzenesulfonamide derivatives disclosed in WO 03053435, R-944, Ro-03-34649, VX-385, GS-224338, OPT-TL3, PL-100, SM-309515, AG-148, DG-35-VIII, DMP-850, GW-5950×, KNI-1039, L-756423, LB-71262, LP-130, RS-344, SE-063, UIC-94-003, Vb-19038, A-77003, BMS-182193, BMS-186318, SM-309515, JE-2147, GS-9005.

Compounds useful as inhibitors of the HIV reverse transcriptase enzyme that may be used in combination with the compounds of the present invention include, but are not limited to, abacavir, FTC, GS-840, lamivudine, adefovir dipivoxil, beta-fluoro-ddA, zalcitabine, didanosine, stavudine, zidovudine, tenofovir, amdoxovir, SPD-754, SPD-756, racivir, reverset (DPC-817), MIV-210 (FLG), beta-L-Fd4C (ACH-126443), MIV-310 (alovudine, FLT), dOTC, DAPD, entecavir, GS-7340, emtricitabine, alovudine.

Compounds useful as non-nucleoside inhibitors of the HIV reverse transcriptase enzyme that may be used in combination with the compounds of the present invention include, but are not limited to, efavirenz, HBY-097, nevirapine, TMC-120 (dapivirine), TMC-125, etravirine, delavirdine, DPC-083, DPC-961, TMC-120, capravirine, GW-678248, GW-695634, calanolide, and tricyclic pyrimidinone derivatives as disclosed in WO 03062238.

Compounds useful as CCR5 inhibitors that may be used in combination with the compounds of the present invention include, but are not limited to, TAK-779, SC-351125, SCH-D, UK-427857, PRO-140, and GW-873140 (Ono-4128, AK-602).

Compounds useful as inhibitors of HIV integrase enzyme that may be used in combination with the compounds of the present invention include, but are not limited to, GW-810781, 1,5-naphthyridine-3-carboxamide derivatives disclosed in WO 03062204, compounds disclosed in WO 03047564, compounds disclosed in WO 03049690, and 5-hydroxypyrimidine-4-carboxamide derivatives disclosed in WO 03035076.

Fusion inhibitors for the treatment of HIV that may be used in combination with the compounds of the present invention include, but are not limited to enfuvirtide (T-20), T-1249, AMD-3100, and fused tricyclic compounds disclosed in JP 2003171381.

Other compounds that are useful inhibitors of HIV that may be used in combination with the compounds of the present invention include, but are not limited to, Soluble CD4, TNX-355, PRO-542, BMS-806, tenofovir disoproxil fumarate, and compounds disclosed in JP 2003119137.

Compounds useful in the treatment or management of infection from viruses other than HIV that may be used in combination with the compounds of the present invention include, but are not limited to, acyclovir, fomivirsen, penciclovir, HPMPC, oxetanocin G, AL-721, cidofovir, cytomegalovirus immune globin, cytovene, fomivganciclovir, famciclovir, foscarnet sodium, Isis 2922, KNI-272, valacyclovir, virazole ribavirin, valganciclovir, ME-609, PCL-016.

Compounds that act as immunomodulators and may be used in combination with the compounds of the present invention include, but are not limited to, AD-439, AD-519, Alpha Interferon, AS-101, bropirimine, acemannan, CL246,738, EL10, FP-21399, gamma interferon, granulocyte macrophage colony stimulating factor, IL-2, immune globulin intravenous, IMREG-1, IMREG-2, imuthiol diethyl dithio carbamate, alpha-2 interferon, methionine-enkephalin, MTP-PE, granulocyte colony stimulating sactor, remune, rCD4, recombinant soluble human CD4, interferon alfa-2, SK&F106528, soluble T4 yhymopentin, tumor necrosis factor (TNF), tucaresol, recombinant human interferon beta, and interferon alfa n-3.

Anti-infectives that may be used in combination with the compounds of the present invention include, but are not limited to, atovaquone, azithromycin, clarithromycin, trimethoprim, trovafloxacin, pyrimethamine, daunorubicin, clindamycin with primaquine, fluconazole, pastill, ornidyl, eflornithine pentamidine, rifabutin, spiramycin, intraconazole-R51211, trimetrexate, daunorubicin, recombinant human erythropoietin, recombinant human growth hormone, megestrol acetate, testerone, and total enteral nutrition.

Antifungals that may be used in combination with the compounds of the present invention include, but are not limited to, anidulafungin, C31G, caspofungin, DB-289, fluconzaole, itraconazole, ketoconazole, micafungin, posaconazole, and voriconazole.

Other compounds that may be used in combination with the compounds of the present invention include, but are not limited to, acmannan, ansamycin, LM 427, AR177, BMS-232623, BMS-234475, Cl-1012, curdlan sulfate, dextran sulfate, STOCRINE EL10, hypericin, lobucavir, novapren, peptide T octabpeptide sequence, trisodium phosphonoformate, probucol, and RBC-CD4.

In addition, the compounds of the present invention may be used in combination with anti-proliferative agents for the treatment of conditions such as Kaposi's sarcoma. Such agents include, but are not limited to, inhibitors of metallo-matrix proteases, A-007, bevacizumab, BMS-275291, halofuginone, interleukin-12, rituximab, paclitaxel, porfimer sodium, rebimastat, and COL-3.

The particular choice of an additional agent or agents will depend on a number of factors that include, but are not limited to, the condition of the mammal being treated, the particular condition or conditions being treated, the identity of the compound or compounds of the present invention and the additional agent or agents, and the identity of any additional compounds that are being used to treat the mammal. The particular choice of the compound or compounds of the invention and the additional agent or agents is within the knowledge of one of ordinary skill in the art and can be made without undue experimentation.

The compounds of the present invention may be administered in combination with any of the above additional agents for the treatment of a mammal, such as a human, that is suffering from an infection with the HIV virus, AIDS, AIDS-related complex (ARC), or any other disease or condition which is related to infection with the HIV virus. Such a combination may be administered to a mammal such that a compound or compounds of the present invention are present in the same formulation as the additional agents described above. Alternatively, such a combination may be administered to a mammal suffering from infection with the HIV virus such that the compound or compounds of the present invention are present in a formulation that is separate from the formulation in which the additional agent is found. If the compound or compounds of the present invention are administered separately from the additional agent, such administration may take place concomitantly or sequentially with an appropriate period of time in between. The choice of whether to include the compound or compounds of the present invention in the same formulation as the additional agent or agents is within the knowledge of one of ordinary skill in the art.

The compounds of the present invention can be prepared by procedures known to those of ordinary skill in the art, those described herein, and those described in co-pending U.S. patent application Ser. Nos. 10/166,957 (filed Jun. 11, 2002), 10/166,979 (filed Jun. 11, 2002), 10/729,645 (filed Dec. 4, 2003), 10/728,602 (filed Dec. 4, 2003), 60/504,018 (filed Sep. 17, 2003), 60/527,470 (filed Dec. 4, 2003), 60/591,354 (filed Jul. 26, 2004), and 60/527,477 (filed Dec. 4, 2003), all of which are hereby incorporated by reference for this purpose. Ritonavir and delavirdine are commercially available or can be prepared by one of ordinary skill in the art using methods known in the art.

### EXAMPLES

In the following examples, both (4R)-N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide and 4,4-difluoro-1-{(2S,3S)-2-hydroxy-3-[(3-hydroxy-2-methylbenzoyl)amino]-4-phenylbutanoyl}-3,3-dimethyl-N-(2,2,2-trifluoroethyl)-L-prolinamide were administered in the form of a solid, spray-dried dispersion comprising 90 wt % of compound and 10 wt % hydroxypropyl methyl cellulose acetate succinate (HPMCAS). Spray-dried dispersions comprising (4R)-N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide, 4,4-difluoro-1-{(2S,3S)-2-hydroxy-3-[(3-hydroxy-2-methylbenzoyl)amino]-4-phenylbutanoyl}-3,3-dimethyl-N-(2,2,2-trifluoroethyl)-L-prolinamide, and N-ethyl-4,4-difluoro-1-{(2S,3S)-2-hydroxy-3-[(3-hydroxy-2,5-dimethylbenzoyl)amino]-4-phenylbutanoyl}-3,3-dimethyl-L-prolinamide can be prepared by methods known to those of ordinary skill in the art, those described herein and those described in co-pending U.S. patent application Ser. Nos. 11/008325, filed Dec. 8, 2004, and 60/591,351, filed Jul. 26, 2004, all of which are hereby incorporated by reference for this purpose.

### Example 1

### Administration of (4R)-N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide (Compound A) Alone or in Combination with 10-Hydroxy-2-methyl-5-(1-methylethyl)-1-[2-(1-methylethyl)-4-thiazolyl]-3,6-dioxo-8,11-bis(phenylmethyl)-2,4,7,12-tetraazatridecan-13-oic acid, 5-thiazolylmethyl Ester, [5S-(5R*,8R*,10R*,11R*)] (ritonavir)

A total of 30 human subjects were administered a single 100 mg, 300 mg, 800 mg, 1000 mg, 1500 mg, 2000 mg, or 3000 mg dose of a spray-dried composition comprising 90 wt % amorphous Compound A and 10 wt % hydroxypropyl methyl cellulose acetate succinate (HPMCAS), under fed conditions according to the schedule found in Table I. Additionally, a total of 12 human subjects were administered a combination of 400 mg or 800 mg of a spray-dried composition, comprising 90 wt % amorphous Compound A and 10 wt % hydroxypropyl methyl cellulose acetate succinate (HPMCAS), and ritonavir, according to the following: 1) at t=0 hours, a 100 mg dose of ritonavir; 2) at t=12 hours, spray-dried composition comprising 90 wt % amorphous Compound A and 10 wt % hydroxypropyl methyl cellulose acetate succinate (HPMCAS), and a 100 mg dose of ritonavir; and 3) at t=24 hours, a 100 mg dose of ritonavir.

Plasma samples were taken from each subject at the following predetermined times: predose (0 hr) and at 0.5, 1, 1.5, 2, 3, 4, 5, 6, 8, 12, 16, 24, 36, and 48 hours postdose. Each plasma sample was analyzed for the presence of Compound A and the following pharmacokinetic parameters for Compound A were determined for each subject: area under the concentration-time curve from time 0 extrapolated to infinity (AUC), maximum observed plasma concentration (Cₘₐₓ), time to first occurrence of Cₘₐₓ (Tₘₐₓ), terminal phase plasma half-life (t_{1/2}), and cumulative amount of drug recovered unchanged in the urine expressed as a percentage of administered dose (Ae %). Pharmacokinetic data for the administration of Compound A alone or in combination with ritonavir are presented in Table I, below.

TABLE I
Summary of Compound A Pharmacokinetics After
Oral Administration of a Single Dose With and Without
Ritonavir (100 mg Every 12 Hours for 3 Doses) Under Fed Conditions

| | | AUC | | | | |
|---|---|---|---|---|---|---|
| Compound A | | (μg × | Cₘₐₓ | | | |
| Dose (mg) | N | (h/mL)^{a} | (μg/mL)^{a} | Tₘₐₓ (h)^{b} | t½ (h)^{c} | Ae %^{c} |
| 100 | 6 | 0.74 | 0.16 | 3.00 | 8.63^{d} | 1.34 |
| 300 | 6 | 3.56 | 0.72 | 2.50 | 8.17 | 2.04 |
| 800 | 6 | 15.8 | 3.12 | 3.00 | 11.7^{e} | 4.00 |
| 1000 | 6 | 16.9 | 3.35 | 3.00 | 7.28 | 4.21 |
| 1500 | 6 | 41.9 | 7.04 | 4.00 | 9.17 | ND |
| 2000 | 6 | 44.2 | 7.49 | 2.00 | 10.4 | 7.92 |
| 3000 | 6 | 86.4 | 11.2 | 4.00 | 7.60 | 9.05 |
| 400 + ritonavir^{f} | 6 | 42.5 | 5.21 | 3.51 | 8.56 | 20.4 |
| 800 + ritonavir^{f} | 6 | 71.4 | 7.35 | 4.00 | 8.39 | 19.4 |

Note:
ND = not determined, Ae % = cumulative amount of drug recovered unchanged in the urine expressed as a percent of administered dose, AUC = area under the concentration-time curve from time 0 to infinity after dosing, Cₘₐₓ = maximum observed drug concentration after single-dose administration, Tₘₐₓ = time to reach Cₘₐₓ, t_{1/2} = terminal phase plasma half-life.
^{a}Geometric means
^{b}Medians
^{c}Arithmetic means
^{d}N = 3 (t½ values for 3 subjects were deemed unreliable and therefore excluded from summary)
^{e}N = 5 (t½ value for 1 subject was deemed unreliable and therefore excluded from summary)
^{f}Compound A was given with the 2^{nd} dose of ritonavir (100 mg every 12 hours for 3 doses)

The mean to t_{1/2} value for the 100 mg dose group was calculated from three subjects. The t_{1/2} values for three subjects at this dose level were deemed unreliable owing to poor estimates of the terminal phase rate constant from regressions (r² values less than 0.9) and, therefore, were not included in the summary statistics. Similarly, the th for one subject in the 800 mg dose group was deemed unreliable owing to poor estimate of the terminal phase rate constant from regression (r² values less than 0.9) and therefore was not included in the summary statistics.

### Example 2

### Administration of 4,4-difluoro-1-{(2S,3S)-2-hydroxy-3-[(3-hydroxy-2-methylbenzoyl)amino]-4-phenylbutanoyl}-3,3-dimethyl-N-(2,2,2-trifluoroethyl)-L-prolinamide (Compound B) Alone or in Combination with 10-Hydroxy-2-methyl-5-(1-methylethyl)-1-[2-(1-methylethyl)-4-thiazolyl]-3,6-dioxo-8,11-bis(phenylmethyl)-2,4,7,12-tetraazatridecan-13-oic Acid, 5-thiazolylmethyl ester, [5S-(5R*,8R*,10R*,11R*)] (ritonavir)

A total of 21 human subjects were administered a single 300 mg, 900 mg, 1800 mg, or 3600 mg dose of a spray-dried composition comprising 90 wt % amorphous Compound B and 10 wt % hydroxypropyl methyl cellulose acetate succinate (HPMCAS) under fasting conditions. Additionally, a total of 12 human subjects were administered a combination of 300 mg or 450 mg of a spray-dried composition, comprising 90 wt % amorphous Compound B and 10 wt % hydroxypropyl methyl cellulose acetate succinate (HPMCAS), and ritonavir with a low-fat/low-calorie meal, according to the following: 1) at t=0 hours, a 100 mg dose of ritonavir; 2) at t=12 hours, spray-dried composition comprising 90 wt % amorphous Compound B and 10 wt % hydroxypropyl methyl cellulose acetate succinate (HPMCAS), and a 100 mg dose of ritonavir; and 3) at t=24 hours, a 100 mg dose of ritonavir. Plasma samples were taken from each patient at pre-dose (O h) and at 0.5, 1, 1.5, 2, 3, 4, 6, 8, 10, 12, 16, 24, 36, and 48 hours after dosing. Each plasma sample was analyzed for the presence of Compound B and the following pharmacokinetic parameters for Compound B were determined for each subject: area under the concentration-time curve from time 0 extrapolated to infinity (AUC), maximum observed plasma concentration (Cₘₐₓ), time to first occurrence of Cₘₐₓ (Tₘₐₓ), terminal phase plasma half-life (t %), CL/F=apparent oral clearance, and cumulative amount of drug recovered unchanged in the urine expressed as a percentage of administered dose (Ae %). Pharmacokinetic data for the administration of Compound B alone or in combination with ritonavir are presented in Table II below.

TABLE II
Summary of Compound B Pharmacokinetics After Oral Administration
of a Single Dose Alone Under Fasting Conditions and With
Ritonavir (100 mg Every 12 Hours for 3 Doses) Under Fed Conditions

| Compound B | Compound B | | AUC | | | | | |
|---|---|---|---|---|---|---|---|---|
| Dose | active dose | | (μg · | Cₘₐₓ | CL/F | Tₘₐₓ | t_{1/2} | |
| (mg) | (mg)^{a} | N | h/mL)^{b} | (μg/mL)^{b} | (L/h)^{b} | (h)^{c} | (h)^{d} | Ae %^{d} |
| 300 | 270 | 5 | 6.11 | 1.53 | 44.2 | 0.50 | 2.76 | 3.67 |
| 900 | 810 | 5 | 28.2 | 4.61 | 28.7 | 2.00 | 3.25 | 4.89 |
| 1800 | 1620 | 6 | 74.5 | 8.67 | 21.7 | 3.02 | 3.60 | 10.4 |
| 3600 | 3240 | 5 | 164 | 14.5 | 19.7 | 4.00 | 4.15 | 11.8 |
| 300 + | 270 + ritonavir^{e} | 6 | 34.8 | 3.10 | 7.75 | 4.00 | 8.54 | 17.7 |
| ritonavir^{e} | | | | | | | | |
| 450 + | 405 + ritonavir^{e} | 6 | 51.6 | 3.83 | 7.84 | 4.02 | 10.3 | 17.6 |
| ritonavir^{e} | | | | | | | | |

Note:
Ae % = cumulative amount of drug recovered unchanged in the urine expressed as a percent of administered dose, AUC = area under the concentration-time curve from time 0 to infinity after dosing, CL/F = apparent oral clearance, Cₘₐₓ, = maximum observed drug concentration after single-dose administration, Tₘₐₓ = time to reach Cₘₐₓ, t_{1/2} = terminal phase plasma half-life.
^{a}Active dose is 90% of the total dose
^{b}Geometric mean
^{c}Median
^{d}Arithmetic mean
^{e}Compound B was given with the 2^{nd} dose of ritonavir (100 mg every 12 hours for 3 doses)

In the examples described below, unless otherwise indicated, all temperatures in the following description are in degrees Celsius (° C.) and all parts and percentages are by weight, unless indicated otherwise.

Various starting materials and other reagents were purchased from commercial suppliers, such as Aldrich Chemical Company or Lancaster Synthesis Ltd., and used without further purification, unless otherwise indicated.

The reactions set forth below were performed under a positive pressure of nitrogen, argon or with a drying tube, at ambient temperature (unless otherwise stated), in anhydrous solvents. Analytical thin-layer chromatography was performed on glass-backed silica gel 60° F. 254 plates (Analtech (0.25 mm)) and eluted with the appropriate solvent ratios (v/v). The reactions were assayed by high-pressure liquid chromotagraphy (HPLC) or thin-layer chromatography (TLC) and terminated as judged by the consumption of starting material. The TLC plates were visualized by UV, phosphomolybdic acid stain, or iodine stain. ¹H-NMR spectra were recorded on a Bruker instrument operating at 300 MHz and ¹³CNMR spectra were recorded at 75 MHz. NMR spectra are obtained as DMSO-d₆ or CDCl₃ solutions (reported in ppm), using chloroform as the reference standard (7.25 ppm and 77.00 ppm) or DMSO-d₆ ((2.50 ppm and 39.52 ppm)). Other NMR solvents were used as needed. When peak multiplicities are reported, the following abbreviations are used: s=singlet, d=doublet, t=triplet, m=multiplet, br=broadened, dd=doublet of doublets, dt=doublet of triplets. Coupling constants, when given, are reported in Hertz. Infrared spectra were recorded on a Perkin-Elmer FT-IR Spectrometer as neat oils, as KBr pellets, or as CDCl₃ solutions, and when reported are in wave numbers (cm⁻¹). The mass spectra were obtained using LC/MS or APCl. All melting points are uncorrected. All final products had greater than 95% purity (by HPLC at wavelengths of 220 nm and 254 nm).

In the following examples and preparations, “Et” means ethyl, “Ac” means acetyl, “Me” means methyl, “Ph” means phenyl, (PhO)₂POCl means chlorodiphenylphosphate, “HCl” means hydrochloric acid, “EtOAc” means ethyl acetate, “Na₂CO₃” means sodium carbonate, “NaOH” means sodium hydroxide, “NaCl” means sodium chloride, “NEt₃” means triethylamine, “THF” means tetrahydrofuran, “DIC” means diisopropylcarbodiimide, “HOBt” means hydroxy benzotriazole, “H₂O” means water, “NaHCO₃” means sodium hydrogen carbonate, “K₂CO₃” means potassium carbonate, “MeOH” means methanol, “i-PrOAc” means isopropyl acetate, “MgSO₄” means magnesium sulfate, “DMSO” means dimethylsulfoxide, “AcCl” means acetyl chloride, “CH₂Cl₂” means methylene chloride, “MTBE” means methyl t-butyl ether, “DMF” means dimethyl formamide, “SOCl₂” means thionyl chloride, “H₃PO₄” means phosphoric acid, “CH₃SO₃H” means methanesulfonic acid, “Ac₂O” means acetic anhydride, “CH₃CN” means acetonitrile, and “KOH” means potassium hydroxide.

### Example 3

### Preparation of (4R)-4-allylcarbamoyl-5,5-dimethyl-thiazolidine-3-carboxylic Acid tert-butyl Ester

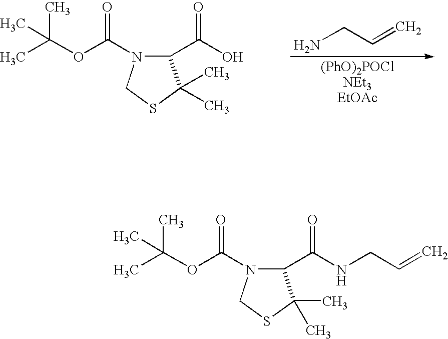

(4R)-5,5-Dimethyl-thiazolidine-3,4-dicarboxylic acid 3-tert-butyl ester (which can be prepared according to the methods of Ikunaka, M. et al., Tetrahedron Asymm. 2002, 13, 1201; Mimoto, T. et al., J. Med. Chem. 1999, 42, 1789; and Mimoto, T. et al., European Patent Application 0574135A1 (1993), 250 g; 0.957 mol) was added to an argon-purged 5-L flask and was dissolved in EtOAc (1.25 L). The solution was cooled to 2° C. and (PhO)₂POCl (208 mL; 1.00 mol) was then added in one portion. NEt₃ (280 mL; 2.01 mol) was added dropwise via addition funnel and the resulting suspension was then stirred at 0° C. Seven minutes later, allylamine (75.4 mL; 1.00 mol) was added dropwise. The ice bath was removed and the suspension was allowed to warm to room temperature. One-half hour later, 1 N HCl (750 mL; 0.750 mol) was added. The mixture was transferred to a 4-L separatory funnel using EtOAc (50 mL) for rinsing. The layers were separated. The organic fraction was washed with 7.2% aqueous Na₂CO₃ (2×1.25 L), and was then transferred to a 3-L distillation flask and was diluted with EtOAc (400 mL). The solution was dried azeotropically and concentrated to a volume of 800 mL by distillation of EtOAc at one atmosphere. After cooling to 25° C., the resulting clear yellowish EtOAc solution of (4R)-4-allylcarbamoyl-5,5-dimethyl-thiazolidine-3-carboxylic acid tert-butyl ester was carried on directly into the next step. An aliquot was removed and concentrated to give (4R)-4-allylcarbamoyl-5,5-dimethyl-thiazolidine-3-carboxylic acid tert-butyl ester as a white crystalline solid: mp=94-98° C., ¹H NMR (300 MHz, CDCl₃) δ 6.12 (br s, 1H), 5.88 (app ddt, J=10.2, 17.1, 5.6 Hz, 1H), 5.28 (app dq, J=17.1, 1.5 Hz, 1H), 5.18 (app dd, J=1.2, 10.2 Hz, 1H), 4.68 (s, 2H), 4.14 (br s, 1H), 3.95 (br t, J=5.4 Hz, 2H), 1.62 (s, 3H), 1.49 (s, 9H), 1.46 (s, 3H); ¹³C NMR (75 MHz, CDCl₃) δ 170.0, 154.0, 134.4, 116.9, 82.0, 73.3, 54.0, 48.7, 42.0, 30.6, 28.6, 24.6; MS (Cl) m/z 301.1599 (301.1586 calcd for C₁₄H₂₅N₂O₃S, M+H⁺); elemental analysis calcd for C₁₄H₂₄N₂O₃S: C, 55.97; H, 8.05; N, 9.32; found: C, 56.11; H, 8.01; N, 9.11.

### Example 4

### Preparation of (4R)-5,5-dimethyl-thiazolidine-4-carboxylic Acid Allylamide

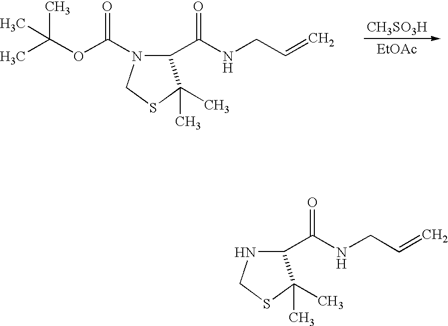

Methanesulfonic acid (155 mL; 2.39 mol) was added dropwise to the EtOAc solution of (4R)-4-allylcarbamoyl-5,5-dimethyl-thiazolidine-3-carboxylic acid tert-butyl ester in a 3-L flask. After stirring at room temperature overnight, the solution was cooled to 7° C. and H₂O (400 mL) was poured in. The mixture was transferred to a 4-L separatory funnel [using H₂O (30 mL) for rinsing] and the layers were separated. The organic fraction was extracted with H₂O (190 mL). The combined H₂O extracts were transferred to a 5-L flask and were cooled to 8° C. The pH was adjusted from 0.4 to 9.3 using 3 N NaOH (−1.05 L). 2-Methyltetrahydrofuran (1.55 L) was poured in, followed by the addition of NaCl (150 g). The ice bath was removed and the mixture was allowed to warm to room temperature. The pH was readjusted to 9.0 using 3 N NaOH (−1 mL). The mixture was transferred to a 4-L separatory funnel, using 2-methyltetrahydrofuran (50 mL) for rinsing, and the layers were separated. The aqueous phase was extracted with 2-methyltetrahydrofuran (950 mL). The organic extracts were vacuum-filtered through Celite directly into a 5-L distillation flask, using 2-methyltetrahydrofuran (200 mL) for rinsing. The solution was dried azeotropically and concentrated to a volume of 1.2 L by distillation of 2-methyltetrahydrofuran at one atmosphere. A measured aliquot was concentrated and weighed, which showed that 161 g of (4R)-5,5-Dimethyl-thiazolidine-4-carboxylic acid allylamide was present in solution [84% from (4R)-5,5-dimethyl-thiazolidine-3,4-dicarboxylic acid 3-tert-butyl ester]. This solution was then carried on directly into the next step. The concentrated aliquot from above yielded (4R)-5,5-Dimethyl-thiazolidine-4-carboxylic acid allylamide as a crystalline solid: mp=45-47° C., ¹H NMR (300 MHz, CDCl₃) δ 6.73 (br s, 1H), 5.87 (app ddt, J=10.2, 17.1, 5.7 Hz, 1H), 5.17-5.27 (m, 2H), 4.27 (AB q, J_{AB}=9.7 Hz, Δv=22.5 Hz, 2H), 2.94 (app tt, J=1.5, 5.8 Hz, 2H), 3.51 (s, 1H), 1.14 (s, 3H), 1.38 (s, 3H); ¹³C NMR (75 MHz, CDCl₃) δ 169.7, 134.4, 116.9, 74.8, 57.2, 51.6, 41.9, 29.1, 27.3; MS (Cl) m/z 201.1063 (201.1062 calcd for C₉H₁₇N₂OS, M+H⁺); elemental analysis calcd for C₉H₁₆N₂OS: C, 53.97; H, 8.05; N, 13.99; found: C, 53.93; H, 8.09; N, 14.07.

### Example 5

### Preparation of (2S,3S)-3-(3-acetoxy-2-methyl-benzoylamino)-2-hydroxy-4-phenyl-butyric Acid

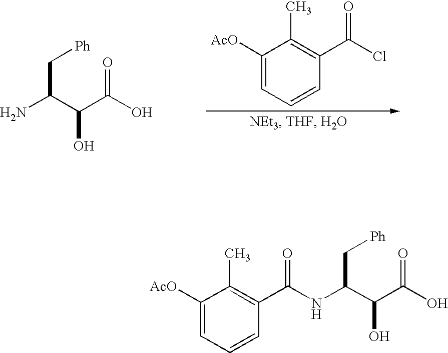

(2S,3S)-3-Amino-2-hydroxy-4-phenyl-butyric acid (which can be prepared according to the method of Pedrosa et al., Tetrahedron Asymm. 2001, 12, 347; M. Shibasaki et al., Tetrahedron Lett. 1994, 35, 6123; and Ikunaka, M. et al. Tetrahedron Asymm. 2002, 13, 1201; 185 g; 948 mmol) was added to a 5-L flask and was suspended in THF (695 mL). H₂O (695 mL) was poured in, followed by NEt₃ (277 mL; 1990 mmol). After stirring for 45 min, the solution was cooled to 6° C. A solution of acetic acid 3-chlorocarbonyl-2-methyl-phenyl ester (201 g; 948 mmol) in THF (350 mL) was then added dropwise. One-half hour later, the pH was adjusted from 8.7 to 2.5 with 6 N HCl (˜170 mL). Solid NaCl (46 g) was added, the ice bath was then removed and the mixture was stirred vigorously while warming to room temperature. The mixture was transferred to 4-L separatory funnel, using 1:1 THF/H₂O (50 mL) for the transfer, and the lower aqueous phase was then removed. The organic fraction was transferred to a 5-L distillation flask, and was then diluted with fresh THF (2.5 L). The solution was azeotropically dried and concentrated to a volume of 1.3 L by distillation of THF at one atmosphere. To complete the azeotropic drying, fresh THF (2.0 L) was added and the solution was concentrated to 1.85 L by distillation at one atmosphere and was then held at 55° C. n-Heptane (230 mL) was added dropwise via addition funnel and the solution was then immediately seeded. After crystallization had initiated, additional n-heptane (95 mL) was added dropwise. The resulting crystal slurry was stirred vigorously for 7 min. Additional n-heptane (1.52 L) was then added as a slow stream. The crystal slurry was then allowed to cool to room temperature slowly and stir overnight. The suspension was vacuum-filtered and the filter cake was then washed with 1:1 THF/n-heptane (700 mL). After drying in a vacuum oven at 45-50° C., 324 g (92%) of (2S,3S)-3-(3-acetoxy-2-methyl-benzoylamino)-2-hydroxy-4-phenyl-butyric acid was obtained as a crystalline solid contaminated with ˜7 mol % Et₃N.HCl: mp=189-191° C., ¹H NMR (300 MHz, DMSO-d₆) δ 12.65 (br s, 1H), 3.80 (d, J=9.7 Hz, 1H), 7.16-7.30 (m, 6H), 7.07 (dd, J=1.1, 8.0 Hz, 1H), 7.00 (dd, J=1.1, 7.5 Hz), 4.40-4.52 (m, 1H), 4.09 (d, J=6.0 Hz, 1H), 2.92 (app dd, J=2.9, 13.9 Hz, 1H), 2.76 (app dd, J=11.4, 13.9 Hz, 1H), 2.29 (s, 3H), 1.80 (s, 3H); ¹³C NMR (75 MHz, DMSO-d₆) δ 174.4, 169.3, 168.1, 149.5, 139.7, 139.4, 129.5, 128.3, 127.9, 126.5, 126.3, 124.8, 123.3, 73.2, 53.5, 35.4, 20.8, 12.6; MS (Cl) m/z 372.1464 (372.1447 calcd for C₂₀H₂₂NO₆, M+H⁺); elemental analysis calcd for C₂₀H₂₁NO₆.0.07 Et₃N.HCl: C, 64.34; H, 5.86; N, 3.95; Cl, 0.70; found: C, 64.27; H, 5.79; N, 3.96; Cl; 0.86.

### Example 6

### Preparation of Acetic Acid 3-{(1S,2S)-3-[(4R)-4-allylcarbamoyl-5,5-dimethyl-thiazolidin-3-yl]-1-benzyl-2-hydroxy-3-oxo-propylcarbamoyl}-2-methyl-phenyl Ester

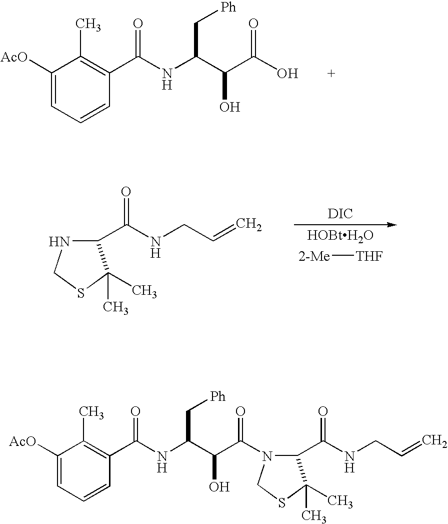

(2S,3S)-3-(3-Acetoxy-2-methyl-benzoylamino)-2-hydroxy-4-phenyl-butyric acid (271 g; 731 mmol) was added to a 5-L flask containing a solution of (4R)-5,5-Dimethyl-thiazolidine-4-carboxylic acid allylamide (161 g; 804 mmol) in 2-methyltetrahydrofuran (1.20 L total solution), while using 2-methyltetrahydrofuran (500 mL) for rinsing. HOBt.H₂O (32.6 g; 241 mmol) was added, using 2-methyltetrahydrofuran (50 mL) for rinsing. The white suspension was allowed to stir at room temperature for 10 min. Diisopropylcarbodiimide (119 mL; 760 mmol) was added in three portions (40 mL+40 mL+39 mL) at 30 min intervals. One hour after the final DIC addition, Celite (100 g) was added and the suspension was allowed to stir at room temperature for 3 h. The mixture was vacuum-filtered, while 2-methyltetrahydrofuran (400 mL) was used to rinse over the solids and wash the resulting filter cake. The filtrate was transferred to 4-L separatory funnel, using 2-methyltetrahydrofuran (50 mL) for rinsing. The solution was washed with 1 N HCl (1.25 L), and then with an aqueous solution of NaHCO₃ (27 g), NaCl (134 g) and H₂O (1.25 L). The resulting organic phase was transferred to a 3-L distillation flask and the solution was then reduced to a volume of 1.12 L by distillation of 2-methyltetrahydrofuran at one atmosphere. The solution was then diluted with 2-methyltetrahydrofuran (230 mL) to bring the total volume to 1.35 L. After cooling the solution to 23° C., the solution of crude acetic acid 3-{(1S,2S)-3-[(4R)-4-allylcarbamoyl-5,5-dimethyl-thiazolidin-3-yl]-1-benzyl-2-hydroxy-3-oxo-propylcarbamoyl}-2-methyl-phenyl ester on directly into the next step.

### Example 7

### Preparation of (4R)-3-[(2S,3S)-2-hydroxy-3-(3-hydroxy-2-methyl-benzoylamino)-4-phenyl-butyryl]-5,5-dimethyl-thiazolidine-4-carboxylic Acid Allylamide

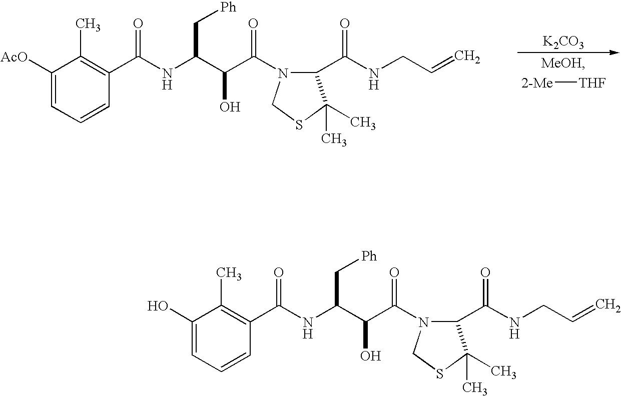

MeOH (330 mL) and K₂CO₃ (66.9 g; 484 mmol) were sequentially added to a 2-methyltetrahydrofuran solution of crude acetic acid 3-{(1S,2S)-3-[(4R)-4-allylcarbamoyl-5,5-dimethyl-thiazolidin-3-yl]-1-benzyl-2-hydroxy-3-oxo-propylcarbamoyl}-2-methyl-phenyl ester (theoretical amount: 405 g; 731 mmol) in a 3-L flask at room temperature. Two and a half hours later, additional K₂CO₃ (20 g; 144 mmol) was added. Three hours later the reaction mixture was vacuum-filtered on a pad of Celite, using 4:1 2-methyltetrahydrofuran/MeOH (330 mL) for rinsing over the solids and washing the filter cake. The filtrate was transferred to a 6-L separatory funnel, using 4:1 2-methyltetrahydrofuran/MeOH (80 mL) for rinsing. The solution was diluted with i-PrOAc (1.66 L) and was then washed with a solution of NaCl (83.0 g) in H₂O (1.60 L). The organic fraction was washed with 0.5 N HCl (1.66 L) and then with a saturated aqueous NaCl solution (400 mL). The resulting organic fraction was transferred to a 4-L Erlenmeyer flask and MgSO₄ (120 g) was added. After stirring for 10 min, the mixture was vacuum-filtered directly into a 5-L distillation flask, using 2:1 i-PrOAc/2-methyltetrahydrofuran (600 mL) for rinsing the separatory funnel and Erlenmeyer flask and washing the MgSO₄. The 2-methyltetrahydrofuran was displaced by distillation at one atmosphere with the simultaneous addition of i-PrOAc in five portions (a total of 3.60 L was used), while maintaining a minimum pot volume of ˜2.50 L. The resulting crystallizing mixture was cooled to 75° C. and was held at this temperature for 30 min. The suspension was then allowed to slowly cool to room temperature overnight. The suspension was vacuum-filtered, using i-PrOAc (600 mL) for transferring and washing the crystals. After drying in a vacuum oven at 40° C., 204 g (54% from (2S,3S)-3-(3-Acetoxy-2-methyl-benzoylamino)-2-hydroxy-4-phenyl-butyric acid) of crystalline (4R)-3-[(2S,3S)-2-Hydroxy-3-(3-hydroxy-2-methyl-benzoylamino)-4-phenyl-butyryl]-5,5-dimethyl-thiazolidine-4-carboxylic acid allylamide was obtained. This material was recrystallized as described below.

### Example 8

### Recrystallization of (4R)-3-[(2S,3S)-2-hydroxy-3-(3-hydroxy-2-methyl-benzoylamino)-4-phenyl-butyryl]-5,5-dimethyl-thiazolidine-4-carboxylic Acid Allylamide

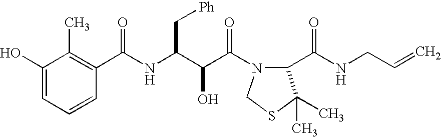

(4R)-3-[(2S,3S)-2-Hydroxy-3-(3-hydroxy-2-methyl-benzoylamino)-4-phenyl-butyryl]-5,5-dimethyl-thiazolidine-4-carboxylic acid allylamide (193 g, 378 mmol) was added to a 5-L flask and was then suspended in EtOAc (1.28 L). After heating the suspension to 76° C., MeOH (68 mL) was added and the internal temperature was then reduced to 70° C. n-Heptane (810 mL) was added dropwise to the solution, while maintaining the internal temperature at 70° C. After the n-heptane addition was complete, the resulting crystal suspension was held at 70° C. for 30 min, and was then allowed to slowly cool to room temperature overnight. The suspension was vacuum-filtered, using 1.6:1 EtOAc/n-heptane (500 mL) to transfer and wash the crystals. The crystals were then dried in a vacuum oven at 45° C. to give 162 g (84% recovery) of purified (4R)-3-[(2S,3S)-2-Hydroxy-3-(3-hydroxy-2-methyl-benzoylamino)-4-phenyl-butyryl]-5,5-dimethyl-thiazolidine-4-carboxylic acid allylamide as a white crystalline solid: mp=173-175° C., ¹H NMR (300 MHz, DMSO-d₆) displayed a ˜10:1 mixture of rotamers, major rotamer resonances δ 9.35 (s, 1H), 8.04-8.15 (m, 2H), 7.13-7.38 (m, 5H), 6.96 (t, J=7.7 Hz, 1H), 6.79 (d, J=7.2 Hz, 1H), 6.55 (d, J=7.5 Hz, 1H), 5.71-5.87 (m, 1H), 5.45 (br d, J=6.2 Hz, 1H), 4.98-5.27 (m, 4H), 4.38-4.52 (m, 3H), 3.58-3.86 (m, 2H), 2.68-2.90 (m, 2H), 1.84 (s, 3H), 1.52 (s, 3H), 1.37 (s, 3H) [characteristic minor rotamer resonances δ 9.36 (s), 8.21 (d, J=10.5 Hz), 7.82 (5, J=5.8 Hz), 4.89 (s), 4.78 (AB q, J_{AB}=9.8 Hz, Δv=27.1 Hz), 4.17-4.24 (m), 2.93-3.01 (m), 1.87 (s), 1.41 (s)]; ¹³C NMR (75 MHz, DMSO-d₆) displayed a ˜10:1 mixture of rotamers, major rotamer resonances δ 170.4, 169.5, 168.2, 155.7, 139.6, 139.4, 135.5, 135.4, 129.9, 128.2, 126.2, 126.1, 121.9, 117.8, 115.6, 72.4, 72.1, 53.1, 51.4, 48.2, 41.3, 34.2, 30.5, 25.0, 12.6 [characteristic minor rotamer resonances δ 171.4, 169.7, 168.6, 139.0, 129.5, 128.4, 70.6, 54.2, 49.1, 41.5, 31.4, 24.8]; MS (Cl) m/z 512.2224 (512.2219 calcd for C₂₇H₃₄N₃O₅S, M+H⁺), elemental analysis calcd for C₂₇H₃₃N₃O₅S: C, 63.38; H, 6.50; N, 8.22; found: C, 63.19; H, 6.52; N, 8.10.

### Example 9

### Preparation of (R)-5,5-dimethyl-thiazolidine-4-carboxylic Acid Allylamide; Hydrochloride

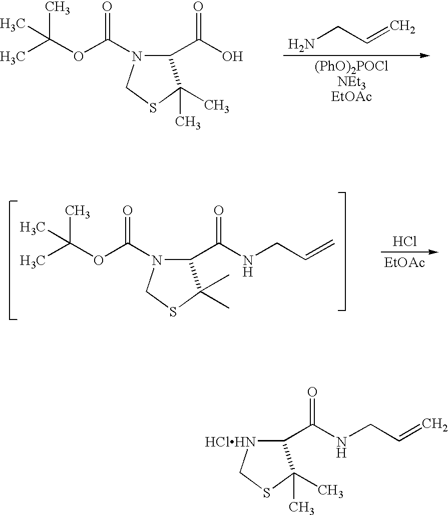

A solution of (R)-5,5-Dimethyl-thiazolidine-3,4-dicarboxylic acid 3-tert-butyl ester (105 kg, 402 mol) and ethyl acetate (690 L) was treated with diphenylchlorophosphate (113 kg, 422 mol) and was then cooled to 0° C. NEt₃ (85.5 kg, 844 mol) was added while maintaining the temperature at 5° C., and the mixture was then held at this temperature for 2 h. The mixture was cooled to 0° C., and allylamine (24.1 kg, 422 mol) was then added while maintaining the temperature at 5° C. The mixture was warmed to 20° C. and was then quenched with 10 wt. % aqueous HCl (310 L). After separation of the layers, the organic fraction was washed with 8.6 wt. % aqueous Na₂CO₃ (710 L). After separation of the layers, the aqueous fraction was extracted with ethyl acetate (315 L). The combined ethyl acetate extracts containing the product were dried by azeotropic distillation at one atmosphere, while maintaining a minimum pot volume of approximately 315 L. The resulting suspension of (R)-4-Allylcarbamoyl-5,5-dimethyl-thiazolidine-3-carboxylic acid tert-butyl ester was cooled to 5° C. A 13 wt. % solution of anhydrous HCl (36.8 kg, 1008 mol) in ethyl acetate (263 L) was cooled to 5° C. and was then added to the (R)-4-Allylcarbamoyl-5,5-dimethyl-thiazolidine-3-carboxylic acid tert-butyl ester suspension while maintaining the temperature at 15° C. The resulting suspension was held at 20° C. for 19 h, and was then cooled and held at 5° C. for 2 h. The suspension was then filtered, using cold ethyl acetate for rinsing. The wet cake was dried under vacuum at 45° C. to give 90.5 kg (95.2%) of (R)-5,5-Dimethyl-thiazolidine-4-carboxylic acid allylamide hydrochloride as a white solid: ¹H NMR (300 MHz, DMSO-d₆) δ 8.94 (app t, J=5.5 Hz, 1H), 5.82 (ddt, J=10.4, 17.2, 5.2 Hz, 1H), 5.19-5.25 (m, 1H), 5.10-5.14 (m, 1H), 4.38 (AB q, J_{AB}=9.8 Hz, Δv=14.5 Hz, 2H), 4.08 (s, 1H), 3.72-3.91 (m, 2H), 1.58 (s, 3H), 1.32 (s, 3H); ¹³C NMR (75 MHz, DMSO-d₆) δ 161.7, 132.2, 114.0, 67.9, 51.4, 43.5, 39.3, 25.3, 24.3; MS (Cl) m/z 201.1070 (201.1062 calcd for C₉H₁₇N₂OS, M+H⁺); elemental analysis calcd for C₉H₁₇ClN₂OS: C, 45.65; H, 7.24; N, 11.83; Cl, 14.97; found: C, 45.41; H, 7.33; N, 11.69; Cl, 15.22.

### Example 10

### Preparation of (2S,3S)-2-acetoxy-3-(3-acetoxy-2-methyl-benzoylamino)-4-phenyl-butyric Acid

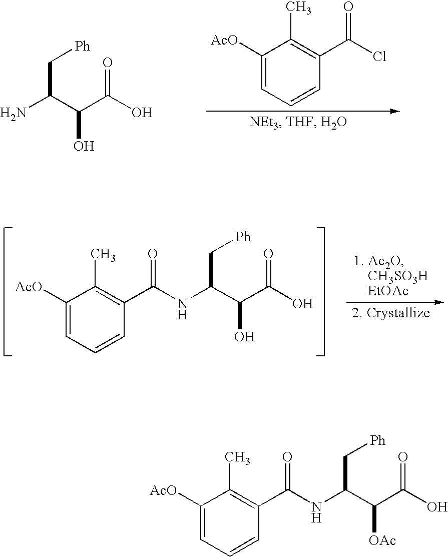

A mixture of (2S,3S)-3-Amino-2-hydroxy-4-phenyl-butyric acid (110 kg, 563 mol), NaCl (195 kg), and THF (413 L) was charged with NEt₃ (120 kg, 1183 mol) and H₂O (414 L) at ambient temperature. The resulting mixture was cooled to 0° C. Acetic acid 3-chlorocarbonyl-2-methyl-phenyl ester (120 kg, 563 mol) was added to a separate reactor and was then dissolved in THF (185 L). The resulting solution of acetic acid 3-chlorocarbonyl-2-methyl-phenyl ester was cooled to 10° C., and was then added to the (2S,3S)-3-amino-2-hydroxy-4-phenyl-butyric acid mixture while maintaining the temperature <10° C. during addition. The resulting biphasic mixture was agitated at 5° C. for 1 h, and was then adjusted to pH 2.5-3.0 with concentrated HCl (62 kg). The mixture was then warmed to 25° C., and the layers were separated. The resulting THF fraction, containing (2S,3S)-3-(3-acetoxy-2-methyl-benzoylamino)-2-hydroxy-4-phenyl-butyric acid, was partially concentrated by distillation at one atmosphere. THF was then replaced with ethyl acetate by distillation at one atmosphere, while maintaining a minimum pot volume of 1500 L. The resulting solution was cooled to 25° C., and was then charged with acetic anhydride (74.8 kg, 733 mol) and methanesulfonic acid (10.8 kg, 112 mol). The mixture was heated at 70° C. for approximately 3 h. The mixture was cooled to 25° C., and was then quenched with H₂O (1320 L) while maintaining the temperature at 20° C. After removal of the aqueous layer, the organic fraction was charged with ethyl acetate (658 L) and H₂O (563 L). After agitation, the aqueous phase was removed. The organic fraction was washed twice with 13 wt. % aqueous NaCl (2×650 L). The organic fraction was partially concentrated and dried by vacuum distillation (70-140 mm Hg) to a volume of approximately 1500 L. The resulting solution was heated to 40° C., and was then charged with n-heptane (1042 L) while maintaining the temperature at 40° C. The solution was seeded with (2S,3S)-2-acetoxy-3-(3-acetoxy-2-methyl-benzoylamino)-4-phenyl-butyric acid (0.1 kg), and additional n-heptane (437 L) was then added slowly. The crystallizing mixture was maintained at 40° C. for 1 h. Additional n-heptane (175 L) was added while maintaining the temperature at 40° C. The crystalline suspension was cooled and held at 25° C. for 1 h, then at 0° C. for 2 h. The suspension was filtered, using n-heptane for rinsing. The wet cake was dried under vacuum at 55° C. to give 174 kg (74.5%) of (2S,3S)-2-acetoxy-3-(3-acetoxy-2-methyl-benzoylamino)-4-phenyl-butyric acid as a white solid: m.p.=152-154° C.; ¹H NMR (300 MHz, CDCl₃) δ 7.21-7.35 (m, 5H), 7.13 (app t, J=7.9 Hz, 1H), 7.01 (app d, J=8.1 Hz, 1H), 6.94 (app d, J=7.2 Hz, 1H), 5.99 (d, J=9.0 Hz, 1H), 5.33 (d, J=4.1 Hz, 1H), 4.96-5.07 (m, 1H), 3.07 (dd, J=5.5, 14.6 Hz, 1H), 2.90 (dd, J=10.0, 14.5 Hz, 1H), 2.30 (s, 3H), 2.18 (s, 3H), 1.96 (s, 3H); ¹³C NMR (125 MHz, CDCl₃) δ 170.4, 170.2, 169.6, 169.5, 149.5, 137.81, 136.5, 129.2, 128.6, 128.4, 127.0, 126.6, 124.5, 123.7, 73.1, 50.9, 35.9, 20.6, 20.5, 12.4; elemental analysis calcd for C₂₂H₂₃NO₇: C, 63.92; H, 5.61; N, 3.39; found: C, 64.22; H, 5.68; N, 3.33; MS (Cl) m/z 414.1572 (414.1553 calcd for C₂₂H₂₄NO₇, M+H⁺).

### Example 11

### Preparation of (4R)-N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide

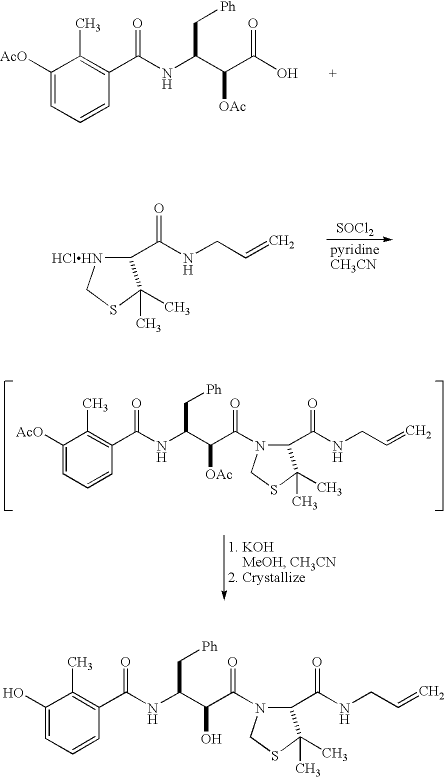

A solution of (2S,3S)-2-acetoxy-3-(3-acetoxy-2-methyl-benzoylamino)-4-phenyl-butyric acid (140 kg, 339 mol), CH₃CN (560 L), and pyridine (64.3 kg, 813 mol) was cooled to 15° C. SOCl₂ (44.3 kg, 373 mol) was charged while maintaining the temperature at 15° C. The mixture was held at 15° C. for 1 h. A separate reactor was charged with (R)-5,5-dimethyl-thiazolidine-4-carboxylic acid allylamide hydrochloride (96.6 kg, 408 mol), CH₃CN (254 L), and pyridine (29.5 kg, 373 mol), and was then cooled to 15° C. The (2S,3S)-2-acetoxy-3-(3-acetoxy-2-methyl-benzoylamino)-4-phenyl-butyric acid chloride solution was added to the (R)-5,5-dimethyl-thiazolidine-4-carboxylic acid allylamide solution, while maintaining the temperature at 15° C. The mixture was held at 15° C. for 6 h. A separate reactor was charged with KOH (167 kg, 2709 mol) and methanol (280 L) using a 0° C. cooling jacket. The resulting KOH/methanol solution was cooled to 5° C. The crude acetic acid 3-{(1S,2S)-2-acetoxy-3-[(R)-4-allylcarbamoyl-5,5-dimethyl-thiazolidin-3-yl]-1-benzyl-3-oxo-propylcarbamoyl}-2-methyl-phenyl ester mixture was added to the KOH/methanol solution while maintaining the temperature at 10° C. After addition was complete, the mixture was held at 25° C. for 3 h. The mixture was charged with H₂O (840 L) and ethyl acetate (840 L), and was then followed by acidification to pH 5-6.5 with concentrated HCl (85 kg) while maintaining the temperature at 20° C. The resulting layers were separated. The organic fraction was sequentially washed with 6.8 wt. % aqueous NaHCO₃ (770 L), an aqueous HCl/NaCl solution (H₂O: 875 L; conc. HCl: 207 kg; NaCl: 56 kg), 8.5 wt. % aqueous NaHCO₃ (322 L), and then with 3.8 wt. % aqueous NaCl (728 L). The resulting organic fraction was partially concentrated by distillation at one atmosphere. The solvent was exchanged with ethyl acetate by continuing distillation and maintaining the pot temperature at ≧70° C. Ethyl acetate was added such that the pot volume remained at approximately 840 L. The solution was then cooled to 20° C. and held at this temperature until crystallization was observed. n-Heptane (280 L) was added and the suspension was agitated at 15° C. for 4 h. The crystals were, using cold 2.4:1 (v/v) ethyl acetate/n-heptane for rinsing. The wet cake was dried under vacuum at 45° C. to provide crude (R)-3-[(2S,3S)-2-hydroxy-3-(3-hydroxy-2-methyl-benzoylamino)-4-phenyl-butyryl]-5,5-dimethyl-thiazolidine-4-carboxylic acid allylamide. Decolorization and recrystallization was conducted as follows: A mixture of crude (R)-3-[(2S,3S)-2-hydroxy-3-(3-hydroxy-2-methyl-benzoylamino)-4-phenyl-butyryl]-5,5-dimethyl-thiazolidine-4-carboxylic acid allylamide, ADP carbon (21 kg), Supercel (3 kg), and ethyl acetate (780 L) was heated to 70° C. CH₃OH (40 L) was added to the mixture. The mixture was filtered, and the resulting clear filtrate was heated to reflux at one atmosphere to begin distillation. CH₃OH was displaced as follows: ethyl acetate (388 L) was charged while maintaining the pot volume at approximately 840 L and at 70° C. The solution was slowly charged with n-heptane (316 L), while maintaining a temperature of 70° C. The mixture was then cooled to 20° C. and was held at this temperature for 4 h. The crystals were filtered, using cold 2.1:1 (v/v) ethyl acetate/n-heptane for rinsing. The wet cake was dried under vacuum at 45° C. to give 103 kg (59.6%) of (4R)-3-[(2S,3S)-2-hydroxy-3-(3-hydroxy-2-methyl-benzoylamino)-4-phenyl-butyryl]-5,5-dimethyl-thiazolidine-4-carboxylic acid allylamide as a white crystalline solid: mp=173-175° C., ¹H NMR (300 MHz, DMSO-d₆) displayed a ˜10:1 mixture of rotamers, major rotamer resonances δ 9.35 (s, 1H), 8.04-8.15 (m, 2H), 7.13-7.38 (m, 5H), 6.96 (t, J=7.7 Hz, 1H), 6.79 (d, J=7.2 Hz, 1H), 6.55 (d, J=7.5 Hz, 1H), 5.71-5.87 (m, 1H), 5.45 (br d, J=6.2 Hz, 1H), 4.98-5.27 (m, 4H), 4.38-4.52 (m, 3H), 3.58-3.86 (m, 2H), 2.68-2.90 (m, 2H), 1.84 (s, 3H), 1.52 (s, 3H), 1.37 (s, 3H) [characteristic minor rotamer resonances δ 9.36 (s), 8.21 (d, J=10.5 Hz), 7.82 (5, J=5.8 Hz), 4.89 (s), 4.78 (AB q, J_{AB}=9.8 Hz, Δv=27.1 Hz), 4.17-4.24 (m), 2.93-3.01 (m), 1.87 (s), 1.41 (s)]; ¹³C NMR (75 MHz, DMSO-d₆) displayed a ˜10:1 mixture of rotamers, major rotamer resonances δ 170.4, 169.5, 168.2, 155.7, 139.6, 139.4, 135.5, 135.4, 129.9, 128.2, 126.2, 126.1, 121.9, 117.8, 115.6, 72.4, 72.1, 53.1, 51.4, 48.2, 41.3, 34.2, 30.5, 25.0, 12.6 [characteristic minor rotamer resonances δ 171.4, 169.7, 168.6, 139.0, 129.5, 128.4, 70.6, 54.2, 49.1, 41.5, 31.4, 24.8]; MS (Cl) m/z 512.2224 (512.2219 calcd for C₂₇H₃₄N₃O₅S, M+H⁺), elemental analysis calcd for C₂₇H₃₃N₃O₅S: C, 63.38; H, 6.50; N, 8.22; found: C, 63.19; H, 6.52; N, 8.10.

### Example 12

### Preparation of (2S,3S)-3-Amino-2-hydroxy-4-phenyl-butyric Acid; Hydrochloride

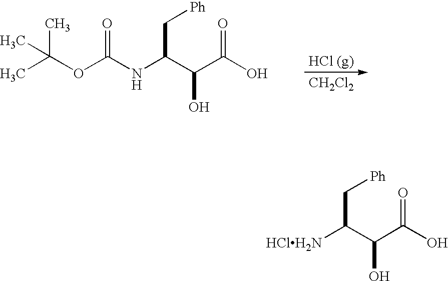

HCl gas (51 g, 1.4 mol) was bubbled into a suspension of (2S,3S)-3-tert-butoxycarbonylamino-2-hydroxy-4-phenyl-butyric acid (163 g, 551 mmol) and CH₂Cl₂ (2.0 L) at 0° C. The resulting off-white suspension was allowed to warm to ambient temperature and stir overnight. ¹H NMR analysis of a concentrated aliquot showed approximately 95% conversion to product. The suspension was cooled to 0° C., and additional HCl gas (46 g, 1.3 mol) was bubbled into the suspension. After warming to ambient temperature, the suspension was stirred overnight. The suspension was vacuum-filtered, the solid was rinsed with CH₂Cl₂ (200 mL), and the solid was then dried in a vacuum oven at 45° C. for 24 h to give 129 g (100%) of (2S,3S)-3-amino-2-hydroxy-4-phenyl-butyric acid; hydrochloride as a white solid: ¹H NMR (300 MHz, DMSO-d₆) δ 13.05 (br s, 1H), 8.25 (br s, 3H), 7.22-7.34 (m, 5H), 4.41 (d, J=2.6 Hz, 1H), 3.66 (br s, 1H), 2.84 (AB portion of ABX, J_{AX}=11.0 Hz, J_{BX}=2.8 Hz, Δv=19.6 Hz, 2H); ¹³C NMR (75 MHz, DMSO-d₆) d 172.4, 136.6, 129.8, 128.7, 127.1, 69.6, 55.0, 33.6; MS (Cl) m/z 196.0979 (196.0974 calcd for C₁₀H₁₄NO₃, M−Cl⁻).

### Example 13

### Preparation of (2S,3S)-3-(3-Acetoxy-2-methyl-benzoylamino)-2-hydroxy-4-phenyl-butyric Acid

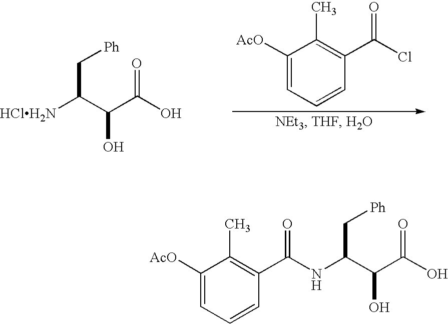

NEt₃ (186 mL, 1.34 mol) was added to a suspension of (2S,3S)-3-amino-2-hydroxy-4-phenyl-butyric acid; hydrochloride (100 g, 432 mmol), H₂O (320 mL), and tetrahydrofuran (320 mL). The suspension was cooled to 4° C. and a solution of acetic acid 3-chlorocarbonyl-2-methyl-phenyl ester (93.6 g, 440 mmol) and THF (160 mL) was added dropwise. The resulting solution was warmed to ambient temperature and stir for 1 h. The solution was cooled to 10° C. and the pH was adjusted to 2.0 using 6 N HCl (87 mL). NaCl (25 g) and tetrahydrofuran (200 mL) were added, and the mixture was warmed to ambient temperature. The phases were separated and the tetrahydrofuran fraction was dried over MgSO₄ and filtered. The filtrate was concentrated to a volume of 330 mL using a rotary evaporator, and was then diluted with tetrahydrofuran (230 mL). n-Heptane (1.2 L) was added slowly and the resulting white suspension of solid was stirred at ambient temperature overnight. The suspension was vacuum-filtered, the solid was rinsed with n-heptane (2×500 mL), and the solid was dried in a vacuum oven at 45° C. for 24 h to give 150 g (93.6%) of (2S,3S)-3-(3-acetoxy-2-methyl-benzoylamino)-2-hydroxy-4-phenyl-butyric acid as a white solid that was contaminated with ˜7.7 mol % Et₃N.HCl: mp=189-191° C., ¹H NMR (300 MHz, DMSO-d₆) δ 12.65 (br s, 1H), 3.80 (d, J=9.7 Hz; 1H), 7.16-7.30 (m, 6H), 7.07 (dd, J=1.1, 8.0 Hz, 1H), 7.00 (dd, J=1.1, 7.5 Hz), 4.40-4.52 (m, 1H), 4.09 (d, J=6.0 Hz, 1H), 2.92 (app dd, J=2.9, 13.9 Hz, 1H), 2.76 (app dd, J=11.4, 13.9 Hz, 1H), 2.29 (s, 3H), 1.80 (s, 3H); ¹³C NMR (75 MHz, DMSO-d₆) δ 174.4, 169.3, 168.1, 149.5, 139.7, 139.4, 129.5, 128.3, 127.9, 126.5, 126.3, 124.8, 123.3, 73.2, 53.5, 35.4, 20.8, 12.6; MS (Cl) m/z 372.1464 (372.1447 calcd for C₂₀H₂₂NO₆, M+H⁺).

### Example 14

### Preparation of a Spray-Dried Dispersion of (4R)-N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide

A spray solution was formed containing 300 g (4R)-N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide, 33.3 g hydroxypropyl methyl cellulose acetate succinate (HPMCAS), and 3000 g methanol as follows. The HPMCAS and methanol were combined in a container and mixed for about 2 hours, allowing the HPMCAS to dissolve. The resulting mixture had a slight haze after the entire amount of polymer had been added. Next, (4R)-N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide was added directly to this mixture, and the mixture stirred for an additional 2 hours. This mixture was then filtered by passing it through a filter with a screen size of 250 μm to remove any large insoluble material from the mixture, thus forming the spray solution.

The spray solution was pumped using a high-pressure pump to a spray drier (a Niro type XP Portable Spray-Dryer with a Liquid-Feed Process Vessel (“PSD-1”)), equipped with a pressure nozzle (Spraying Systems Pressure Nozzle and Body) (SK 76-16). The PSD-1 was equipped with a 9-inch chamber extension. The 9-inch chamber extension was added to the spray dryer to increase the vertical length of the dryer. The added length increased the residence time within the dryer, which allowed the product to dry before reaching the angled section of the spray dryer. The spray drier was also equipped with a 316 SS circular diffuser plate with 1/16-inch drilled holes, having a 1% open area. This small open area directed the flow of the drying gas to minimize product recirculation within the spray dryer. The nozzle sat flush with the diffuser plate during operation. A Bran+Lubbe high-pressure pump was used to deliver liquid to the nozzle. The pump was followed by a pulsation dampener to minimize pulsation at the nozzle. The spray solution was pumped to the spray drier at about 180 g/min at a pressure of 200 psig. Drying gas (e.g., nitrogen) was circulated through the diffuser plate at an inlet temperature of 200° C. The evaporated solvent and drying gas exited the spray drier at a temperature of 60° C. The resulting solid amorphous dispersion was collected in a cyclone. The solid amorphous dispersion formed using the above procedure was post-dried using a Gruenberg single-pass convection tray dryer operating at 40° C. for 6 hours. Following drying, the dispersion was then equilibrated with ambient air and humidity (20° C./50% RH) for 8 hours.

### Example 15

### Preparation of (2S)-4,4-difluoro-3,3-dimethyl-pyrrolidine-2-carboxylic Acid (2,2,2-trifluoro-ethyl)-amide; Hydrochloride

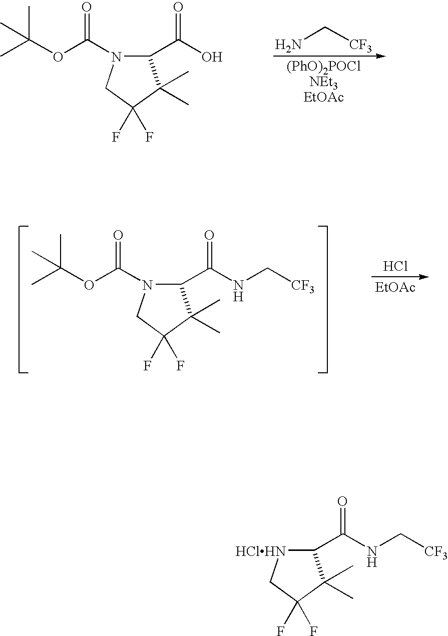

NEt₃ (75.2 g, 743 mmol) was slowly added to a 10° C. solution of (2S)-4,4-difluoro-3,3-dimethyl-pyrrolidine-1,2-dicarboxylic acid 1-tert-butyl ester (98.3 g, 352 mmol), chlorodiphenylphosphate (101 g, 376 mmol), and ethyl acetate (1.0 L). The mixture was warmed to ambient temperature for 45 min., and was then cooled to 10° C. 2,2,2-Trifluoroethylamine (39.5 g, 399 mmol) was slowly added and the resultant mixture was stirred at ambient temperature for 2.75 h. 20% Aqueous citric acid (1.0 L) was added and the resulting layers were separated. The aqueous fraction was extracted with ethyl acetate (2×300 mL). The combined organic fractions were washed with saturated aqueous NaHCO₃ (2×500 mL), and then with saturated aqueous NaCl (300 mL). The resulting organic fraction was concentrated to a weight of 900 g using a rotary evaporator. A 3 N HCl/ethyl acetate solution (500 mL) was added to the concentrate, and the mixture was stirred at ambient temperature for 24 h. The resulting solid was filtered, washed with ethyl acetate (100 mL), and was then dried in a vacuum oven at 55° C. to provide 98.0 g (93.9%) of (2S)-4,4-difluoro-3,3-dimethyl-pyrrolidine-2-carboxylic acid (2,2,2-trifluoro-ethyl)-amide; hydrochloride as a white solid: ¹H NMR (300 MHz, DMSO-d₆) δ 10.46 (br s, 2H), 9.50 (t, J=6.2 Hz, 1H), 4.17-4.33 (m, 2H), 3.68-4.02 (m, 3H), 1.23 (app d, J=2.1 Hz, 3H), 0.97 (app d, J=2.0 Hz, 3H); ¹³C NMR (75 MHz, DMSO-d₆) δ 165.6, 127.9 (dd, J_{CF}=250.2, 257.2 Hz), 125.6 (q, J_{CF}=279.0 Hz), 64.8, 48.2 (t, J_{CF}=33.4 Hz), 45.7 (t, J_{CF}=21.2 Hz), 18.2 (d, J_{CF}=7.5 Hz), 17.2 (app dd, J_{CF}=2.3, 5.8 Hz); MS (Cl) m/z 261.1015 (261.1026 calcd for C₉H₁₄N₂OF₅, M−HCl+H⁺); elemental analysis calcd for C₉H₁₄N₂OClF₅: C, 36.44; H, 4.76; N, 9.44; Cl, 11.95; F, 32.02; found: C, 36.45; H, 4.86; N, 9.43; Cl, 12.06; F, 32.15.

### Example 16

### Preparation of 4,4-difluoro-1-{(2S,3S)-2-hydroxy-3-[(3-hydroxy-2-methylbenzoyl)amino]-4-phenylbutanoyl}-3,3-dimethyl-N-(2,2,2-trifluoroethyl)-L-prolinamide

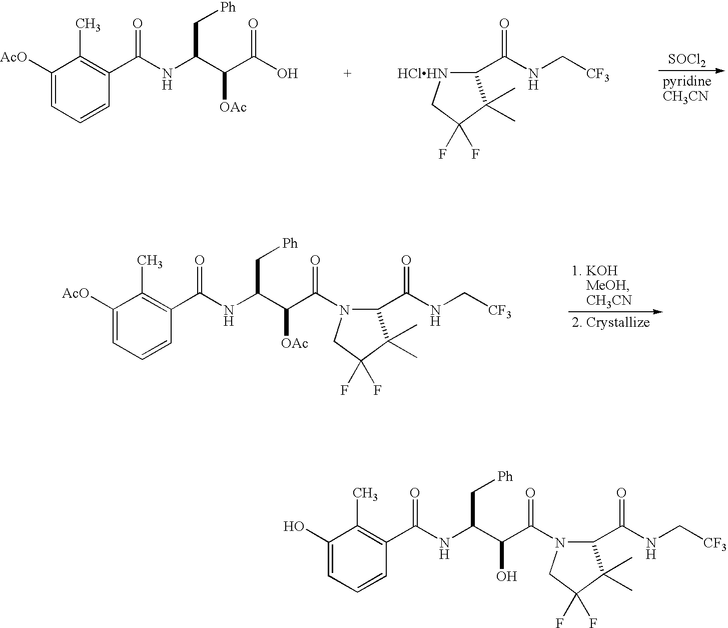

Pyridine (149 g, 1.89 mol) was added to a solution of (2S,3S)-2-acetoxy-3-(3-acetoxy-2-methyl-benzoylamino)-4-phenyl-butyric acid (193 g, 468 mmol) and acetonitrile (1.6 L) at ambient temperature, and the mixture was then cooled to 10° C. A solution of SOCl₂ (62.3 g, 523 mmol) and acetonitrile (50 mL) was added over 15 min., and cooling was then discontinued. 15 minutes later, additional SOC₂ (0.80 g, 6.7 mmol) was added. After stirring at ambient temperature for 25 min., the mixture was cooled to 10° C. (2S)-4,4-Difluoro-3,3-dimethyl-pyrrolidine-2-carboxylic acid (2,2,2-trifluoro-ethyl)-amide; hydrochloride from Synthesis 3 (139 g, 468 mmol) was added in portions over 15 min. The mixture was warmed to ambient temperature for 1 h, and was then cooled to 10° C. A 5° C. solution of KOH (85% assay; 186 g, 2.82 mol) and methanol (1.1 L) was then added over 10 min, followed by addition of K₂CO₃ (51.8 g, 375 mmol). The mixture was warmed to ambient temperature for 1 h, and was then concentrated to a weight of 1.5 kg using a rotary evaporator. The resulting mixture was partitioned between 0.5 N HCl (1.6 L) and ethyl acetate (1.4 L), and the layers were separated. The organic fraction was sequentially washed with saturated aqueous NaHCO₃ (1.4 L), 0.5 N HCl (1.6 L), and then H₂O (1.4 L). The organic fraction was concentrated to a wet solid using a rotary evaporator, and was then further dried in a vacuum oven at 50° C. for 24 h. The resulting solid was dissolved in absolute ethanol (800 mL), and was then concentrated on a rotary evaporator. The resulting solid was once again dissolved in ethanol (600 mL), then concentrated on a rotary evaporator, and then dried in a vacuum oven at 50° C. for 24 h. The solid was dissolved in ethanol and 0.11 N HCl (620 mL) was then slowly added. H₂O (950 mL) was slowly added and the resulting suspension of crystals was stirred overnight. The solid was filtered, washed with ethanol/H₂O (1:3, 200 mL), and dried in a vacuum oven at 55° C. to provide 259 g (96.9%) of the title compound as a solid: ¹H NMR (300 MHz, DMSO-d₆) displayed a ˜20:1 mixture of rotamers. Major rotamer resonances δ 9.34 (s, 1H), 8.66 (app t, J=6.3 Hz, 1H), 8.13 (d, J=8.3 Hz, 1H), 7.15-7.35 (m, 5H), 6.96 (app t, J=7.7 Hz, 1H), 6.79 (d, J=7.3 Hz, 1H), 6.55 (d, J=6.7 Hz, 1H), 5.56 (d, J=6.4 Hz, 1H), 4.26-4.54 (m, 5H), 3.81-4.07 (m, 2H), 2.86-2.90 (m, 1H), 2.71 (app dd, J=10.5, 13.6 Hz, 1H), 1.82 (s, 3H), 1.22 (s, 3H), 1.04 (s, 3H) [characteristic minor rotamer resonances δ 8.62 (5, J=6.5 Hz), 5.35 (d, J=7.6 Hz), 1.86 (s)]; ¹³C NMR (75 MHz, DMSO-d₆) displayed a ˜20:1 mixture of rotamers. Major rotamer resonances δ 171.5, 169.6, 168.6, 155.7, 139.6, 139.4, 129.8, 128.2, 127.9 (dd, J_{CF}=251.7, 253.5 Hz), 126.2, 126.0, 125.0 (q, J_{CF}=279.2 Hz), 121.8, 117.9, 115.6, 73.2, 68.3, 53.0, 51.4 (t, J_{CF}=32.6 Hz), 43.8 (t, J_{CF}=20.8 Hz), 34.5, 22.4 (d, J_{CF}=4.1 Hz), 16.9 (d, J_{CF}=7.3 Hz), 12.5 [characteristic minor rotamer resonances δ 171.7, 139.1, 129.5, 68.7, 47.0 (t), 16.5 (d)]; MS (Cl) m/z 572.2189 (527.2184 calcd for C₂₇H₃₁N₃O₅F₅, M+H⁺); elemental analysis calcd for C₂₇H₃₀N₃O₅F₅: C, 56.74; H, 5.29; N, 7.35; F, 16.62; found: C, 56.50; H, 5.50; N, 7.15; F, 16.36.

### Example 17

### Preparation of a Spray-Dried Dispersion of 4,4-difluoro-1-{(2S,3S)-2-hydroxy-3-[(3-hydroxy-2-methylbenzoyl)amino]-4-phenylbutanoyl}-3,3-dimethyl-N-(2,2,2-trifluoroethyl)-L-prolinamide

A solid amorphous dispersion containing 90 wt % 4,4-difluoro-1-{(2S,3S)-2-hydroxy-3-[(3-hydroxy-2-methylbenzoyl)amino]-4-phenylbutanoyl}-3,3-dimethyl-N-(2,2,2-trifluoroethyl)-L-prolinamide and 10 wt % HPMCAS-M (AQUOT-MG, available from Shin Etsu), was prepared as follows. First, a spray solution was formed containing 39.0 g of 4,4-difluoro-1-{(2S,3S)-2-hydroxy-3-[(3-hydroxy-2-methylbenzoyl)amino]-4-phenylbutanoyl}-3,3-dimethyl-N-(2,2,2-trifluoroethyl)-L-prolinamide, 4.34 g HPMCAS-M, and 390 g methanol as follows. The HPMCAS-M was added to methanol in a container and stirred. Next, 4,4-difluoro-1-{(2S,3S)-2-hydroxy-3-[(3-hydroxy-2-methylbenzoyl)amino]-4-phenylbutanoyl}-3,3-dimethyl-N-(2,2,2-trifluoroethyl)-L-prolinamide was added directly to this mixture, and the mixture stirred for a total of 2 hours. The resulting mixture had a slight haze after all the ingredients had been added and dissolved.

The spray solution was added to a tank and pressurized using compressed nitrogen to pass the solution through an inline filter (140 μm screen size) and then to a pressure-swirl atomizer (Schlick #1 pressure nozzle) located in a spray-drying chamber as described in Example 14. The spray solution was pressurized at a pressure of about 75 psig, at a flow rate of about 10 g/min. Drying gas (nitrogen) entered the spray-drying chamber at a flow of about 400 g/min and an inlet temperature of about 125° C. The evaporated solvent and drying gas exited the spray drier at a temperature of 60° C. The resulting solid amorphous dispersion was collected in a cyclone.

The solid amorphous dispersion formed using the above procedure was post-dried using a Gruenberg single-pass convection tray dryer operating at 40° C./15% RH for 6 hours. Following drying, the dispersion was then equilibrated with ambient air and humidity (20° C./50% RH) for 2 hours.

### Example 18

### Preparation of 3-acetoxy-2,5-dimethyl-benzoic Acid

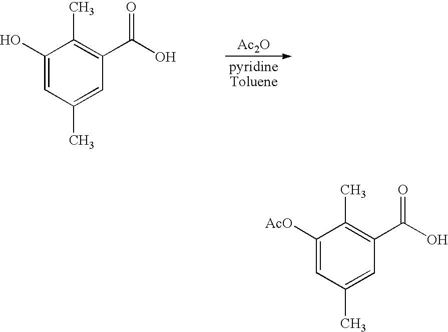

Pyridine (34.0 mL, 419 mmol) and acetic anhydride (150 mL, 1.59 mol) were sequentially added to a suspension of 3-hydroxy-2,5-dimethyl-benzoic acid (211 g, 1.27 mol) in toluene (1.05 L). The mixture was heated at 50° C. under argon for 6 h. Heating was discontinued and, while the mixture was still warm, n-heptane (2.10 L) was added. The mixture was allowed to cool and stir at ambient temperature overnight. The suspension was filtered, using n-heptane for rinsing, and the solid was dried in a vacuum oven at 50° C. to give 212 g (80.1%) of 3-acetoxy-2,5-dimethyl-benzoic acid as a pale yellow solid: m.p.=153-154° C.; ¹H NMR (300 MHz, CDCl₃) δ 11.5 (br s, 1H), 7.80 (s, 1H), 7.10 (s, 1H), 2.44 (s, 3H), 2.41 (s, 3H), 2.39 (s, 3H); ¹³C NMR (75 MHz, DMSO-d₆) δ 169.3, 168.8, 149.9, 136.3, 132.9, 128.4, 128.0, 126.3, 20.8, 20.5, 13.1; MS (Cl) m/z 209.0822 (209.0814 calcd for C₁₁H₁₃O₄, M+H⁺); elemental analysis calcd for C₁₁H₁₂O₄: C, 63.45; H, 5.81; found: C, 63.54; H, 5.88.

### Example 19

### Preparation of Acetic Acid 3-chlorocarbonyl-2,5-dimethyl-phenyl Ester

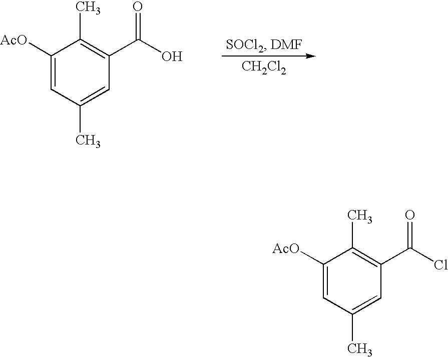

SOCl₂ (80.0 mL, 1.09 mol) was added to a suspension of 3-acetoxy-2,5-dimethyl-benzoic acid (206 g, 990 mmol), DMF (4.0 mL), and CH₂Cl₂ (1.03 L). The resulting mixture was stirred at ambient temperature for 1.5 h. n-Heptane (1.03 L) was added, followed by the slow addition of saturated aqueous NaHCO₃ (2.06 L), and the layers were then separated. The organic fraction was washed with saturated aqueous NaCl (1.00 L), dried over MgSO₄, filtered, and concentrated with a rotary evaporator to give 193 g (86.2%) of acetic acid 3-chlorocarbonyl-2,5-dimethyl-phenyl ester as a pale yellow solid: m.p.=52-54° C.; ¹H NMR (300 MHz, CDCl₃)δ 7.92 (s, 1H), 7.15 (s, 1H), 2.44 (s, 3H), 2.38 (s, 3H), 2.35 (s, 3H); ¹³C NMR (75 MHz, CDCl₃) δ 169.4, 167.7, 150.1, 137.3, 134.7, 132.0, 130.2, 129.1, 21.2, 21.1, 13.7; elemental analysis calcd for C₁₁H₁₁O₃Cl: C, 58.29; H, 4.89; found: C, 58.64; H, 4.89.

### Example 20

### Preparation of (2S,3S)-3-(3-acetoxy-2,5-dimethyl-benzoylamino)-2-hydroxy-4-phenyl-butyric Acid

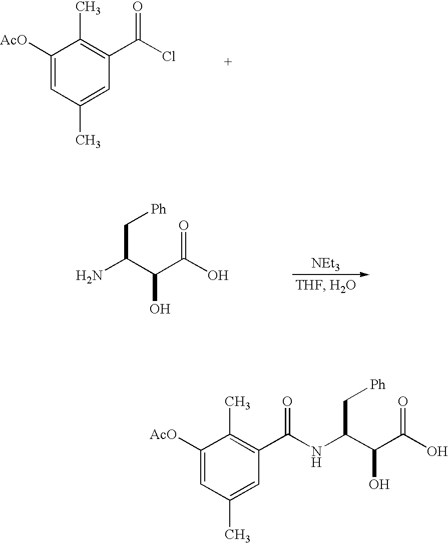

NEt₃ (265 mL, 1.88 mol) was added to a suspension of (2S,3S)-3-amino-2-hydroxy-4-phenyl-butyric acid (175 g, 896 mmol), tetrahydrofuran (875 mL), and H₂O (875 mL) at ambient temperature. The resulting solution was cooled to 0° C. A solution of acetic acid 3-chlorocarbonyl-2,5-dimethyl-phenyl ester (193 g, 854 mmol) and tetrahydrofuran (430 mL) was slowly added. One hour later, H₂O (225 mL) was added, followed by the slow addition of 3 N HCl (390 mL). The resulting mixture was allowed to slowly warm to ambient temperature with stirring overnight. The solid was filtered, using H₂O (430 mL) for rinsing. After drying in a vacuum oven at 50° C., 301 g (91.5%) of (2S,3S)-3-(3-acetoxy-2,5-dimethyl-benzoylamino)-2-hydroxy-4-phenyl-butyric acid was obtained as a white solid that was contaminated with ˜8 mol % Et₃N.HCl: m.p.=220-224° C.; ¹H NMR (300 MHz, DMSO-d₆) δ 12.65 (br s, 1H), 8.23 (d, J=9.0 Hz, 1H), 7.15-7.30 (m, 5H), 6.89 (s, 1H), 6.79 (s, 1H), 5.63 (br s, 1H), 4.39-4.50 (m, 1H), 4.07 (d, J=5.9 Hz, 1H), 2.91 (app dd, J=3.0, 14.0 Hz, 1H), 2.74 (app dd, J=11.1, 14.1 Hz, 1H), 2.27 (s, 3H), 1.24 (s, 3H), 1.72 (s, 3H) [characteristic resonances of Et₃N.HCl: δ 3.09 (q, J=7.3 Hz), 1.18 (t, J=7.3 Hz)]; ¹³C NMR (75 MHz, DMSO-d₆) δ 174.4, 169.2, 168.2, 149.4, 139.4, 135.9, 129.5, 128.3, 126.3, 125.6, 124.7, 123.5, 73.2, 53.5, 35.4, 20.8, 20.6, 12.2 [characteristic resonances of Et₃N.HCl: δ 45.9, 8.8]; MS (Cl) m/z 386.1600 (386.1604 calcd for C₂₁H₂₄NO₆, M+H⁺); elemental analysis calcd for C₂₁H₂₃NO₆.0.08 Et₃N.HCl: C, 65.08; H, 6.17; N, 3.82; found: C, 64.88; H, 6.10; N, 3.68.

### Example 21

### Preparation of (2S,3S)-2-acetoxy-3-(3-acetoxy-2,5-dimethyl-benzoylamino)-4-phenyl-butyric Acid

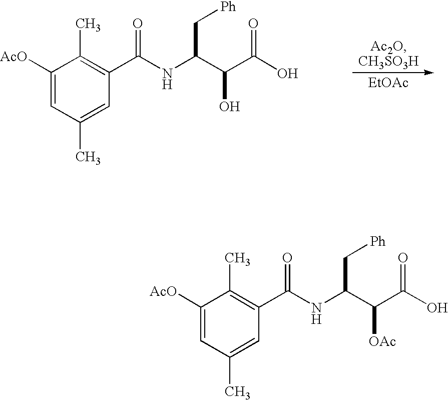

Methanesulfonic acid (16.5 mL, 253 mmol) and acetic anhydride (91.0 mL, 960 mmol) were sequentially added to a suspension of (2S,3S)-3-(3-acetoxy-2,5-dimethyl-benzoylamino)-2-hydroxy-4-phenyl-butyric acid (296 g, 768 mmol) in ethyl acetate (3.00 L) at ambient temperature. The mixture was heated at 75° C. for 2 h, and the resulting solution was then cooled to ambient temperature. The solution was sequentially washed with H₂O (2.0 L), half-saturated aqueous NaCl (2.0 L), and then with saturated aqueous NaCl (1.0 L). The resulting organic fraction was concentrated to approximately half volume by distillation at one atmosphere. Heating was discontinued and the solution was allowed to cool to ambient temperature to give a suspension. n-Heptane (3.0 L) was added and the suspension stirred at ambient temperature overnight. The solid was filtered, using 1:2 ethyl acetate/n-heptane (1.5 L) for rinsing. After drying in a vacuum oven at 50° C., 316 g (96.3%) of (2S,3S)-2-acetoxy-3-(3-acetoxy-2,5-dimethyl-benzoylamino)-4-phenyl-butyric acid was obtained as a white solid: m.p.=185-186° C.; ¹H NMR (300 MHz, DMSO-d₆) δ 13.3 (s, 1H), 8.49 (d, J=8.8 Hz, 1H), 7.19-7.34 (m, 5H), 6.91 (s, 1H), 6.71 (s, 1H), 5.11 (d, J=5.0 Hz, 1H), 4.61-4.72 (m, 1H), 2.79-2.90 (m, 2H), 2.27 (s, 3H), 2.24 (s, 3H), 2.14 (s, 3H), 1.73 (s, 3H); ¹³C NMR (75 MHz, DMSO-d₆) δ 170.3, 169.7, 169.2, 168.5, 149.4, 139.1, 138.5, 136.1, 129.4, 128.5, 126.6, 125.4, 124.7, 123.8, 73.9, 51.1, 35.2, 20.9, 20.8, 20.6, 12.1; MS (Cl) m/z 428.1713 (428.1709 calcd for C₂₃H₂₆NO₇, M+H⁺); elemental analysis calcd for C₂₃H₂₅NO₇: C, 64.63; H, 5.90; N, 3.28; found: C, 64.79; H, 5.96; N, 3.15.

### Example 22

### Preparation of (2S)-4,4-difluoro-3,3-dimethyl-pyrrolidine-2-carboxylic Acid Ethylamide; Hydrochloride

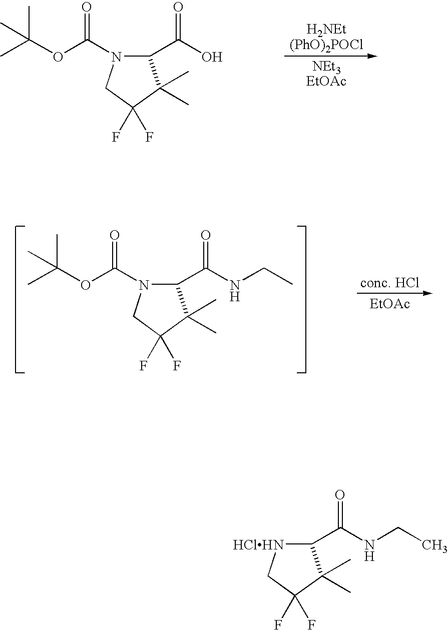

Chlorodiphenylphosphate (38.4 mL, 185 mmol) was added to a solution of (2S)-4,4-difluoro-3,3-dimethyl-pyrrolidine-1,2-dicarboxylic acid 1-tert-butyl ester (48.8 g, 175 mmol) in ethyl acetate (490 mL) at ambient temperature. The solution was cooled to 0° C., and NEt₃ (51.0 mL, 367 mmol) was added dropwise. Cooling was discontinued and the resulting suspension was allowed to warm to ambient temperature and stir for 1 h. The suspension was cooled to 0° C., and H₂NEt (96.0 mL of a 2.0 M solution in tetrahydrofuran, 192 mmol) was slowly added. The resulting mixture was allowed to warm to ambient temperature and stir for 2 h. 20% Aqueous citric acid (490 mL) was added and the layers were then separated. The aqueous fraction was extracted with ethyl acetate (125 mL). The combined organic fractions were washed with saturated aqueous NaHCO₃ (490 mL), and the layers were then separated. The aqueous fraction was extracted with ethyl acetate (125 mL). The combined organic fractions were washed with saturated aqueous NaCl (250 mL), dried over MgSO₄, and then concentrated to a volume of ˜500 mL using a rotary evaporator. Concentrated HCl (61.0 mL, 734 mmol) was added, and the solution was stirred at ambient temperature overnight. The resulting suspension was dried azeotropically with ethyl acetate (3×250 mL) by distillation at one atmosphere. The resulting suspension was cooled to ambient temperature, and was then filtered, using ethyl acetate (100 mL) for rinsing. After drying under vacuum at ambient temperature, 37.4 g (88.2%) of (2S)-4,4-difluoro-3,3-dimethyl-pyrrolidine-2-carboxylic acid ethylamide; hydrochloride was obtained as a white solid: m.p.=238-239° C. (decomp.); ¹H NMR (300 MHz, DMSO-d₆) δ 10.3 (br s, 2H), 8.70 (t, J=5.3 Hz, 1H), 4.08 (s, 1H), 3.71-3.80 (m, 2H), 3.08-3.34 (m, 2H), 1.21 (app d, J=2.2 Hz, 3H), 1.08 (t, J=7.2 Hz, 3H), 0.97 (app d, J=2.1 Hz, 3H); ¹³C NMR (75 MHz, DMSO-d₆) δ 163.8, 128.1 (dd, J_{CF}=248.6, 255.5 Hz), 64.8, 48.1 (t, J_{CF}=33.7 Hz), 45.5 (t, J_{CF}=20.8 Hz), 34.3, 18.3 (d, J_{CF}=7.4 Hz), 17.4 (app dd, J_{CF}=2.1, 5.4 Hz), 14.8; MS (Cl) m/z 207.1317 (207.1309 calcd for C₉H₁₇N₂OF₂, M−HCl+H⁺); elemental analysis calcd for C₉H₁₇ClF₂N₂O: C, 44.54; H, 7.06; N, 11.54; F, 15.66; found: C, 44.40; H, 7.06; N, 11.65; F, 15.61.

### Example 23

### Preparation of Acetic Acid 3-{(1S,2S)-2-acetoxy-1-benzyl-3-[(2S)-2-ethylcarbamoyl-4,4-difluoro-3,3-dimethyl-pyrrolidin-1-yl]-3-oxo-propylcarbamoyl}-2,5-dimethyl-phenyl Ester

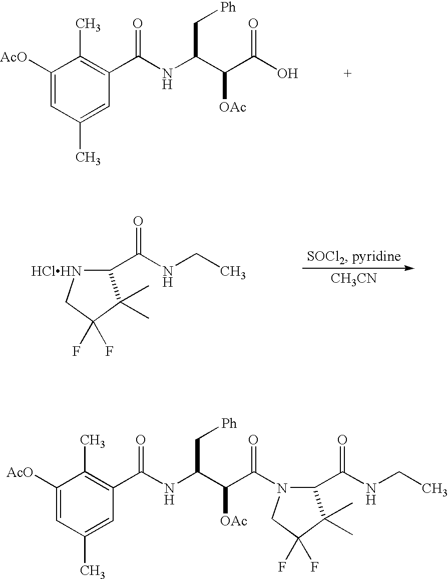

SOCl₂ (1.90 mL, 25.8 mmol) was added dropwise to a 0° C. solution of (2S,3S)-2-acetoxy-3-(3-acetoxy-2,5-dimethyl-benzoylamino)-4-phenyl-butyric acid (10.0 g, 23.5 mmol), pyridine (7.60 mL, 93.9 mmol), and CH₃CN (90.0 mL). The resulting solution was allowed to warm to ambient temperature for 1 h, then was cooled to 0° C. (2S)-4,4-difluoro-3,3-dimethyl-pyrrolidine-2-carboxylic acid ethylamide; hydrochloride (5.71 g, 23.5 mmol) was added in one portion. The resulting solution was allowed to warm to ambient temperature and stir for 2.5 h. Saturated aqueous NaHCO₃ (110 mL) and methyl t-butyl ether (110 mL) were added, and the resulting layers were separated. The resulting organic fraction was sequentially washed with 20% aqueous citric acid (90 mL), saturated aqueous NaHCO₃ (70 mL), and saturated aqueous NaCl (70 mL). Activated charcoal (14 g) was added to the resulting organic fraction, and the mixture was stirred at ambient temperature overnight. The mixture was filtered on Celite, using methyl t-butyl ether for rinsing. The filtrate was dried over MgSO₄, filtered, and concentrated to a volume of ˜90 mL using a rotary evaporator. This solution of crude acetic acid 3-{(1S,2S)-2-acetoxy-1-benzyl-3-[(2S)-2-ethylcarbamoyl-4,4-difluoro-3,3-dimethyl-pyrrolidin-1-yl]-3-oxo-propylcarbamoyl}-2,5-dimethyl-phenyl ester was carried directly to the next step. Analytical data was obtained by concentrating a sample of this solution: m.p.=88-93° C.; ¹H NMR (300 MHz, DMSO-d₆) displayed a ˜10:1 mixture of rotamers. Major rotamer resonances: δ 8.58 (d, J=8.2 Hz, 1H), 8.02 (t, J=7.5 Hz, 1H), 7.18-7.42 (m, 5H), 6.92 (s, 1H), 6.84 (s, 1H), 5.34 (d, J=3.2 Hz, 1H), 4.41-4.66 (m, 2H), 4.19-4.32 (m, 2H), 3.03-3.26 (m, 2H), 2.95 (app dd, J=2.4, 13.8 Hz, 1H), 2.78 (app dd, J=11.7, 13.8 Hz, 1H), 2.27 (s, 3H), 2.25 (s, 3H), 1.73 (s, 3H), 1.22 (br s, 3H), 1.07 (br s, 3H), 1.04 (t, J=7.2 Hz, 3H) [characteristic minor rotamer resonances: δ 7.76-7.87 (m), 6.72 (s), 5.46 (d, J=3.7 Hz), 2.07 (s), 1.79 (s)]; ¹³C NMR (75 MHz, DMSO-d₆) displayed a ˜10:1 mixture of rotamers. Major rotamer resonances: δ 170.5, 169.2, 169.0, 166.8, 166.7, 149.4, 139.1, 138.8, 136.1, 129.7, 128.3, 127.8 (dd, J_{CF}=251.2, 254.9 Hz), 126.5, 125.7, 124.7, 123.9, 73.3, 68.2, 51.4, 43.9 (t, J_{CF}=20.5 Hz), 33.8, 33.4, 22.0 (d, J_{CF}=6.0 Hz), 20.8, 20.5, 17.6 (d, J_{CF}=7.0 Hz), 15.0, 12.2 [characteristic minor rotamer resonances: δ 169.5, 168.9, 167.0, 149.5, 138.7, 129.3, 128.5, 125.4, 124.8, 124.2, 34.1, 21.2, 14.7]; MS (Cl) m/z 616.2859 (616.2834 calcd for C₃₂H₄₀N₃O₇F₂, M+H⁺); elemental analysis calcd for C₃₂H₃₉F₂N₃O₇: C, 62.43; H, 6.38; N, 6.83; F, 6.17; found: C, 62.08; H, 6.68; N, 6.53; F, 5.85.

### Example 24

### Preparation of N-ethyl-4,4-difluoro-1-{(2S,3S)-2-hydroxy-3-[(3-hydroxy-2,5-dimethylbenzoyl)amino]-4-phenylbutanoyl}-3,3-dimethyl-L-prolinamide

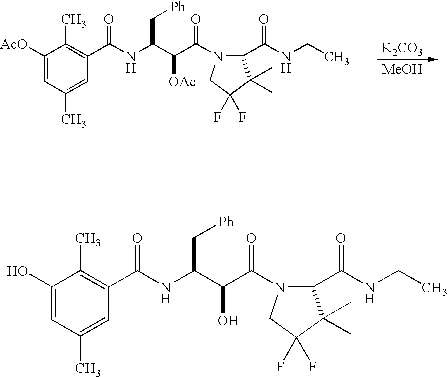

Methanol (30.0 mL) and K₂CO₃ (7.16 g, 51.7 mmol) were added to the methyl t-butyl ether solution of acetic acid 3-{(1S,2S)-2-acetoxy-1-benzyl-3-[(2S)-2-ethylcarbamoyl-4,4-difluoro-3,3-dimethyl-pyrrolidin-1-yl]-3-oxo-propylcarbamoyl}-2,5-dimethyl-phenyl ester (from above) at ambient temperature. After stirring for 2 h, the resulting yellow solution was diluted with ethyl acetate (140 mL), 1 N HCl (50 mL), and 0.5 N HCl (140 mL), and the layers were then separated. The resulting organic fraction was sequentially washed with saturated aqueous NaHCO₃ (90 mL), 0.5 N HCl (70 mL), H₂O (140 mL), and saturated aqueous NaCl (70 mL). The organic fraction was then concentrated to a volume of ˜100 mL by distillation at one atmosphere, and the resulting solution was then cooled to ambient temperature. Diisopropyl ether (190 mL) was slowly added, and the resulting crystalline suspension was stirred overnight at ambient temperature. The suspension was filtered, using diisopropyl ether (50 mL) for rinsing. After drying under vacuum, 9.88 g (79.1%) of N-ethyl-4,4-difluoro-1-{(2S,3S)-2-hydroxy-3-[(3-hydroxy-2,5-dimethylbenzoyl)amino]-4-phenylbutanoyl}-3,3-dimethyl-L-prolinamide was obtained as a white solid: m.p.=208-214° C.; ¹H NMR (300 MHz, DMSO-d₆) displayed a ˜9:1 mixture of rotamers. Major rotamer resonances: δ 9.21 (s, 1H), 8.07 (d, J=8.2 Hz, 1H), 7.90 (t, J=5.5 Hz, 1H), 7.15-7.39 (m, 5H), 6.62 (s, 1H), 6.40 (s, 1H), 5.45 (d, J=6.3 Hz, 1H), 3.95-4.50 (m, 5H), 3.02-3.22 (m, 2H), 2.89 (app dd, J=2.0, 13.5 Hz, 1H), 2.72 (app dd, J=10.4, 13.4 Hz, 1H), 2.17 (s, 3H), 1.78 (s, 3H), 1.22 (s, 3H), 1.05 (s, 3H), 1.03 (t, J=7.2 Hz, 3H) [characteristic minor rotamer resonances: δ 6.15 (d, J=8.7 Hz), 7.85 (t, J=5.7 Hz), 6.34 (s), 5.31 (d, J=7.6 Hz), 4.73 (s), 1.81 (s); ¹³C NMR (75 MHz, DMSO-d₆) displayed a ˜9:1 mixture of rotamers. Major rotamer resonances: δ 171.0, 169.6, 167.2, 155.5, 139.7, 139.1, 135.1, 129.8, 128.2, 128.1 (dd, J_{CF}=251.4, 254.0 Hz), 126.2, 118.7, 118.6, 116.2, 72.8, 68.5, 53.1, 51.5 (t, J_{CF}=32.0 Hz), 43.7 (t, J_{CF}=20.5 Hz), 34.2, 33.8, 22.5 (d, J_{CF}=4.7 Hz), 20.9, 17.4 (d, J_{CF}=7.3 Hz), 15.1, 12.2 [characteristic minor rotamer resonances: δ 171.8, 169.7, 168.0, 138.8, 129.5, 23.1, 14.9; MS (Cl) m/z 532.2614 (532.2623 calcd for C₂₈H₃₆N₃O₅F₂, M+H⁺); elemental analysis calc for C₂₈H₃₅F₂N₃O₅: C, 63.26; H, 6.64; N, 7.90; F, 7.15; found: C, 63.20; H, 6.67; N, 7.87; F, 7.07.

### Example 25

### Preparation of a Spray-Dried Dispersion of N-ethyl-4,4-difluoro-1-{(2S,3S)-2-hydroxy-3-[(3-hydroxy-2,5-dimethylbenzoyl)amino]-4-phenylbutanoyl}-3,3-dimethyl-L-prolinamide

A solid amorphous dispersion containing 90 wt % N-ethyl-4,4-difluoro-1-{(2S,3S)-2-hydroxy-3-[(3-hydroxy-2,5-dimethylbenzoyl)amino]-4-phenylbutanoyl}-3,3-dimethyl-L-prolinamide and 10 wt % HPMCAS-M (AQOAT-MG, available from Shin Etsu), was prepared as follows. First, a spray solution was formed containing 9.0 wt % N-ethyl-4,4-difluoro-1-{(2S,3S)-2-hydroxy-3-[(3-hydroxy-2,5-dimethylbenzoyl)amino]-4-phenylbutanoyl}-3,3-dimethyl-L-prolinamide, 1.0 wt % HPMCAS-M, and 90.0 wt % methanol as follows. The HPMCAS-M was added to methanol in a container and stirred. Next, N-ethyl-4,4-difluoro-1-{(2S,3S)-2-hydroxy-3-[(3-hydroxy-2,5-dimethylbenzoyl)amino]-4-phenylbutanoyl}-3,3-dimethyl-L-prolinamide was added directly to this mixture, and the mixture stirred for a total of 2 hours. The resulting mixture had a slight haze after all the ingredients had been added and dissolved.

The spray solution was added to a tank and pressurized using compressed nitrogen to pass the solution through an inline filter (140 μm screen size) and then to a pressure-swirl atomizer (Schlick #1 pressure nozzle) located in a spray-drying chamber as described in Example 14. The spray solution was pressurized at a pressure of about 75 psig, at a flow rate of about 10 g/min. Drying gas (nitrogen) entered the spray-drying chamber at a flow of about 400 g/min and an inlet temperature of about 125° C. The evaporated solvent and drying gas exited the spray drier at a temperature of 60° C. The resulting solid amorphous dispersion was collected in a cyclone. The solid amorphous dispersion formed using the above procedure was post-dried using a Gruenberg single-pass convection tray dryer operating at 40° C./5% RH for a minimum of 10 hours.

1. A method for improving the pharmacokinetics of (4R)-N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide in a mammal, comprising administering to said mammal (4R)-N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide, or a pharmaceutically acceptable salt or solvate thereof, and at least one agent that inhibits the enzyme activity of cytochrome P450.

2. A method according to claim 1, wherein said (4R)-N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide, or a pharmaceutically acceptable salt or solvate thereof, is administered to said mammal in an amount from about 100 mg to about 2000 mg.

3. A method according to claim 1, wherein said at least one agent that inhibits the enzyme activity of cytochrome P450 is chosen from ritonavir and delavirdine.

4. A method according to claim 3, wherein said at least one agent that inhibits the enzyme activity of cytochrome P450 is ritonavir.

5. A method according to claim 4, wherein said ritonavir is administered to said mammal in an amount from about 10 mg to about 500 mg.

6. A method according to claim 3, wherein said at least one agent that inhibits the enzyme activity of cytochrome P450 is delavirdine.

7. A method according to claim 1, wherein said (4R)-N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide, or a pharmaceutically acceptable salt or solvate thereof, and at least one agent that inhibits the enzyme activity of cytochrome P450, are present in an amount sufficient that (4R)-N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide is present in the plasma of said mammal in a concentration of from about 0.001 μM to about 10 μM for at least about 6 hours after said administration.

8. A method according to claim 7, wherein said plasma concentration of said (4R)-N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide is from about 0.01 μM to about 5 μM for at least about 6 hours after said administration.

9. A method according to claim 1, wherein said cytochrome P450 is the 3A4 isoform.

10. A method according to claim 1, wherein said (4R)-N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide, or a pharmaceutically acceptable salt or solvate thereof, and said at least one agent that inhibits the enzyme activity of cytochrome P450 are present in the same unitary dosage form.

11. A composition comprising (4R)-N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide, or a pharmaceutically acceptable salt or solvate thereof, and at least one agent that inhibits the enzyme activity of cytochrome P450.

12. A composition according to claim 11, wherein said cytochrome P450 is the 3A4 isoform.

13. A composition according to claim 11, wherein said at least one agent that inhibits the enzyme activity of cytochrome P450 is chosen from ritonavir and delavirdine.

14. A composition according to claim 13, wherein said at least one agent that inhibits the enzyme activity of cytochrome P450 is ritonavir.

15. A composition according to claim 14, wherein said (4R)-N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide, or a pharmaceutically acceptable salt or solvate thereof, and said ritonavir are present in a ratio of from about 1:1 to about 20:1 by weight.

16. A composition according to claim 14, wherein said (4R)-N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide, or a pharmaceutically acceptable salt or solvate thereof, and said ritonavir are present in a ratio of from about 1:1 to about 10:1 by weight.

17. A composition according to claim 14, wherein said (4R)-N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide, or a pharmaceutically acceptable salt or solvate thereof, and said ritonavir are present in a molar ratio of from about 1:0.1 to about 10:1.
